(19) United States
(12) Patent Application Publication
(10) Pub. No.: US 2014/0335162 A1
(43) Pub. Date: Nov. 13, 2014

(54) PHARMACEUTICAL COMPOSITION FOR TREATMENT OF ALLERGIC REACTIONS

(71) Applicants: PLS Design GmbH, Hamburg (DE); HELMHOLTZ ZENTRUM MUNCHEN FORSCHUNGSZENTRUM FUR GESUNDHEIT UND UMWELT GMBH, Neuherberg (DE)

(72) Inventors: Thomas Grunwald, Hamburg (DE); Carsten B. Schmidt-Weber, Munchen (DE)

(21) Appl. No.: 14/360,343

(22) PCT Filed: Nov. 23, 2012
(86) PCT No.: PCT/EP2012/004884

§ 371 Date: May 23, 2014

(30) Foreign Application Priority Data
Nov. 23, 2011 (EP) 11075261.5

(51) Int. Cl. A61K 39/35; A61K 47/34; A61K 38/20

(52) U.S. Cl. CPC A61K 39/35; A61K 38/2026; A61K 47/34; A61K 2039/577
USPC 424/450; 424/85.2; 424/486

(57) ABSTRACT

The invention relates to a pharmaceutical composition made of one or more preparations and comprising a physiologically effective dose of at least one IL-4 and/or IL-13 inhibitor and at least one allergene, and a matrix, wherein at least the inhibitor is solved or embedded, or whereon at least the inhibitor is coated or adsorbed, wherein the matrix is selected as to enable prolonged release of the inhibitor.

### FIELD OF THE INVENTION

The present invention relates to methods for coincident in vivo blockade of IL-4- and IL-13-mediated pathways along with allergen stimulation of T cells to augment the induction of clinical tolerance to allergens.

### BACKGROUND OF THE INVENTION AND STATE OF THE ART

Allergic diseases including immediate type (type I) hypersensitivity result from an unbalanced response of the specific immune system. In type-I hypersensitivity, Th2 cells upregulate CD40 ligand on their surface upon contact with an allergen-presenting cell. Interaction of CD40 ligand with the CD40 on B cells is accompanied by the production of Th2-related cytokines like IL-4, IL-5 and IL-13 and a subsequent class-switching of the immunoglobulins leads to the production of IgE antibodies which mediate various clinical symptoms.

However, the existence of allergen-specific Th2 cells is not sufficient for the development of allergy since these cells are also found in healthy individuals. Critical for allergy pathogenesis is the number of allergen-specific regulatory T cells (Treg) capable of suppressing the proliferation and cytokine expression of Th1 and Th2 cells, and acting on antigen-presenting cells (APC).

Regulatory T cells are classified broadly as natural T regulatory T cells (nTreg) and induced or adaptive regulatory T cells (iTreg). The natural regulatory T cells are self antigen specific CD4⁺T cells that express FOXP3 transcription factor and high amounts of CD25. They are selected in the thymus and become regulatory T cell in the periphery. Naïve CD4⁺Th cells in the periphery can develop into regulatory T cells and, therefore, are called induced or adaptive regulatory T cells. The expression of FOXP3 transcription factor is considered a key factor for the induction of regulatory T cells These cells including type 1 regulatory T cells (Tr1) and Th3 cells can produce IL-10 or TGF-beta or both, by which they exert most of their suppressive activity (for reviews, see Schmidt-Weber, 2005; Nandakumar et al., 2009).

The suppression of the immune response by regulatory T cells occurs by various pathways (for reviews, see Schmidt-Weber, 2005; Rouse, 2007; Nandakumar et al., 2009). IL-10 directly and indirectly suppresses the activity of mast cells, basophils and eosinophils. It also promotes IgG4. TGF-beta is a switch and promotion factor for B cells towards IgA isotype and thereby controls peripheral tolerance. Most important, TGF-beta has been shown to promote FOXP3 expression and to inhibit the Th1-driving transcription factor T-BET as well as the Th2-driving transcription factor GATA-3. IL-10 has also been shown to promote FOXP3 expression in vitro, but only if IL-4 and IL-12 were neutralized. Both cytokines represent decision signals for T-cell differentiation, and induce the transcription factors GATA-3 or T-BET, respectively.

Generally, Treg-mediated suppression appears to be antigen specific, although the responsible cytokines are secreted and, thereby, potentially are available for a variety of T cells. However, recent studies have shown that cytokines and cytokine receptors can become part of the immunological synapse that is formed between the APCs and the T cells (for a review, see Schmidt-Weber, 2005). Thereby, soluble cytokines can be shuttled in an antigen-specific manner to a neighbouring cell and Tregs can switch a particular target cell into a non-responsive, non-apoptotic state (anergy).

Among the cytokines contributing to the development of allergic inflammation IL-4 and IL-13 play key roles in the pathogenesis of asthma and other allergic diseases. Both cytokines represent pleiotropic cytokines and are associated with the induction of the IgE isotype switch, secretion of IgE by B lymphocytes, and enhancement of IgE-mediated responses by up-regulating IgE receptors on B lymphocytes, mast cells and basophils. However, IL-4 and IL-13 have also some distinct non-overlapping functions. For example, IL-4 is able to drive the differentiation of naïve Th cells into Th2 lymphocytes, leading to the production of effector cytokines such as IL-4, IL-5, IL-9, and IL-13. IL-13 has important effects on epithelial cell maturation, mucus production, generation of extracellular matrix proteins, enhancement of the contractility of airway smooth muscle cells, and the recruitment of eosinophils, macrophages and T cells.

IL-4 exerts its activities by interacting with specific Type I and type II receptors on the cell surface (for a review, see Gessner et al., 2000). The type I receptor comprises a 140 kDa binding chain (Swiss Prot accession number P24394), IL-4Rα (also referred to as CD 124), and the common γ-chain (γ_{c}) of the IL-2R, which is shared by multiple cytokine receptors. In the absence of the common γ-chain IL-4 can use the type II IL-4 receptor, comprising IL-4Rα and an IL-13 binding chain, IL-13Rα1. This receptor complex is also a functional receptor for IL-13, and this explains the overlap of the majority of biological effects of IL-4 and IL-13, although both cytokines exhibit only 25% homology. Type I receptor complexes can only be formed by IL-4 and appear to be responsible for mediating IL-4 responses in T cells which do not express IL-13Rα1. Type II receptor complexes can be formed and activated by either IL-4 or IL-13. The type I receptor complex predominates in hematopoietic cells, whereas the type II receptor complex is expressed on both hematopoietic cells and non-hematopoietic cells. The binding sequence of IL-4 and IL-13 to type II receptor complexes differs in that IL-4 first binds to IL-4Rα with high affinity before associating with the second subunit, whereas IL-13 first binds to IL-13Rα1 with low affinity and then this complex recruits IL-4Rα to form a high affinity binding site.

A second IL-13 binding protein, IL-13 receptor α2 (IL-13Rα2), binds IL-13 with high affinity but does not bind IL-4. However, IL-13Rα2 is believed to be non-signaling, thereby serving as a decoy receptor. It has been shown that treatment of epithelial cells with IFN-γ increases surface expression of IL-13Rα2 through mobilization from intracellular stores, suggesting that IL-13Rα2 plays a key role in responding to and regulating the Th1/Th2 balance by competing with the IL-4Rα/IL-13Rα1 complex for IL-13.

Soluble IL-4 receptors (sIL-4Rα) lacking the transmembrane and intracellular domains are present in biological fluids. While the precise function of sIL-4Rα remains to be elucidated, it may even enhance IL-13 responses by forming a complex with IL-13Rα1 on the cell surface, thereby stabilizing the weak interaction of IL-13 with IL-13Rα1. When tested in bronchial fibroblasts, the presence of sIL-4Rα enhanced IL-13-stimulated eotaxin production in response to suboptimal doses of IL-13. In addition, sIL-4Rα may act as a carrier protein from which IL-4 can dissociate over time. Therey, sIL-4Rα could offer some kind of protection of IL-4 against proteolytic degradation.

### Specific Immunotherapy

Specific allergen immunotherapy (IT) is the only widely used treatment capable of restoring clinical tolerance to allergen and is associated with the re-induction of allergen-specific Treg cells. Peripheral tolerance to allergen in the normal immune response of healthy individuals to allergen exposure and in specific immunotherapy is mainly the result of sufficient Treg numbers. Therefore, the main principle of specific immunotherapy in atopic patients is to increase the number of Tregs capable of controlling allergen-specific effector T cells.

Successful IT is associated with a change of T cell memory from the pro-allergic Th2 phenotype towards a peripheral IL-10-producing T cell phenotype and a local phenotype that also shows FOXP3 expression in addition to IL-10 positive cells. It was previously shown that immunotherapy can result in long-term benefit with disease remission for 3 years following discontinuation. However, considering the various factors influencing the efficacy of specific immunotherapeutic approaches, it is not surprising that the clinical outcome varies to a great extent. Immunotherapies are efficient when patients are monosensibilized against seasonal allergens, but can be less or not efficient if the patient is atopic or if the patient reacts to perennial allergens.

One problem appears to be the quantity of individual allergens in allergen extracts derived from natural sources since in some extracts important allergens are not present in sufficient amounts. However, successful specific immunotherapy requires application of high allergen doses which promote the engagement of the low-affinity FcεRII (CD23) known as a negative feedback regulator of IgE-dependent responses. If present in sufficient quantities the administered allergen will not only be bound by specific IgE, but also by other immunoglobulin classes. IgG complexes with allergen are important in modulating the immune response and preventing IgE-mediated reactions. The existence of three types of Fcγ-receptors is known. FcγRI and FcγRIII, expressed primarily on cells of the myeloid lineage, can mediate pro-inflammatory functions such as phagocytosis, antibody-dependent cytotoxicity and the release of inflammatory mediators. FcγRII expressed on both myeloid and lymphoid cells, transmits inhibitory signals into the cells. Activated dendritic cells are potent T-cell stimulators and express both inhibitory and activatory FcγRs. In view of these facts the recent development or recombinant allergens for specific immunotherapy appears to be a promising approach since they allow the design of allergen compositions containing all important allergens in optimal concentrations. In addition, recombinant allergens allow site-directed modifications of their surface structure in order to decrease the density of B cell epitopes. The aim of such allergen modification is to decrease the allergenicity while retaining its immunogenicity. Evaluation of modified recombinant allergens with a strongly reduced IgE reactivity that display the full spectrum of linear T cell epitopes but a different surface structure as compared to the corresponding natural allergen, have demonstrated that such molecules are capable of reducing specific IgE development towards the native allergen (Niederberger et al., 2004; Karamloo et al., 2005).

However, despite recent improvements the efficacy of specific immunotherapy needs to be further optimized. Appropriate approaches for increasing the number of Tregs capable of controlling allergen-specific effector T cells remain to be defined. Therefore, there is a need in art to define critical factors influencing the induction of Tregs and to design more effective allergen-tolerogenic therapies.

### Technical Problem of the Invention

Thus, the technical problem of the invention is to provide means for treatment of allergic reactions, wherein the induction of clinical tolerance to allergenes is improved, in particular wherein the systemic side effects of IL-3 and/or IL-13 inhibitor administration are reduced and in combination therewith the stimulation of T-cells for balancing the response of the specific immune system is enhanced. Specifically, the technical problem is to provide a pharmaceutical composition, uses thereof, a method for making such composition and a treatment method for achieving these goals.

### SUMMARY OF THE INVENTION

For solving these technical problems the invention provides the subject matters defined in the claims. The invention is explained in more detail in the following.

Peripheral tolerance to allergen in the normal immune response of healthy individuals to allergen exposure and in specific immunotherapy is mainly the result of sufficient Treg numbers. Therefore, the main principle of specific immunotherapy in atopic patients is to increase the number of Tregs capable of controlling allergen-specific effector T cells. Under in vitro conditions, the allergen and the absence of Th1- or Th2-driving factors have been demonstrated to be essential for the induction of Tregs. Since IL-4 and IL-13 play key roles in the development of allergic inflammation, the present invention provides methods for coincident in vivo inhibition of IL-4- and IL-13-mediated effects along with allergen stimulation of T cells to augment the induction of clinical tolerance to allergens.

Preferred inhibitors of IL-4- and IL-13-mediated effects suitable for the method of the present invention include but are not limited to a) antagonistic IL-4 and IL-13 derivatives, b) soluble Il-4 and IL-13 receptor constructs, c) monoclonal antibodies, fragments thereof or other proteinaceous constructs with specificity for IL-4 and IL-13 capable of blocking their biologic activity, d) monoclonal antibodies, fragments thereof or other proteinaceous constructs with specificity for IL-4 and IL-13 receptor complexes capable of blocking the interaction of IL-4 and IL-13 with their receptor subunits, e) aptamers with specificity for IL-4 and IL-13 capable of blocking their biologic activity, and f) aptamers with specificity for IL-4 and IL-13 receptor complexes capable of blocking the interaction of IL-4 and IL-13 with their receptor subunits.

In one embodiment, the present invention discloses methods for a prolonged inhibition of IL-4- and IL-13-mediated effects at the site of allergen presentation along with a prolonged presentation of allergens. To achieve effective and prolonged inhibition of IL-4- and IL-13-mediated effects at the site of allergen presentation, the present invention discloses methods for a sustained release of such inhibitors or combinations of such inhibitors from a depot at the site of allergen presentation.

In a specific embodiment, the present invention discloses matrices for prolonged delivery of inhibitors of decision signals for T-cell differentiation. Preferred matrices include but are not limited to biodegradable polymers which are suitable as depot for substantial quantities of such inhibitors or inhibitor combinations, which allow the release of sufficient quantities of the inhibitor or inhibitor combinations for efficient local inhibition of IL-4- and IL-13-mediated pathways over a prolonged period of time, and which are chemically and physically compatible with the adjuvant and allergen (s) used for the induction of tolerance according to the method of the present invention.

In a preferred specific embodiment, injectable in situ-forming gel systems which are biodegradable, are used for controlled delivery of inhibitors of IL-4- and IL-13-mediated pathways. Such in situ-forming gel systems (hydrogels) undergo a sol-gel-sol transition, which is free flowing sol at room temperature and a non-flowing gel at body temperature. Compared to other biodegradable polymers, the injectable thermogelling polymers possess several advantages including easy preparation, high encapsulation efficiency of bioactive molecules such as inhibitors of IL-4- and IL-13-mediated pathways, and free of harmful organic solvents in the formulation process.

In another embodiment, the present invention discloses methods for restricting high local concentrations of inhibitors of IL-4- and IL-13-mediated effects mainly to the site of allergen presentation to reduce adverse effects due to interaction of the inhibitors with targets distal from the site of allergen presentation. In one specific embodiment, inhibitors of IL-4- and IL-13-mediated effects are used which provide a relatively short serum half-life that is sufficient for the inhibitors to be locally active upon their release from a depot at the site of allergen presentation, and which allows fast removal from circulation upon diffusion and transport away from the site of allergen presentation. Preferred inhibitors providing such characteristics include but are not limited to a) antagonistic IL-4 and IL-13 derivatives with a molecular weight of ≦15 kDa, b) small antibody fragments or other proteinaceous constructs capable of inhibiting IL-4- and IL-13-mediated effects, and c) small molecular weight aptamers capable of inhibiting IL-4- and IL-13-mediated effects. In another specific embodiment, inhibitors of IL-4- and IL-13-mediated effects are used which are susceptible to inactivation by endogeneous enzymes after their release from a depot at the site of allergen presentation and which can be derivatized to adjust their susceptibility to enzymatic inactivation according to the needs of the method of the present invention. Preferred inhibitors providing such characteristics include but are not limited to aptamers the susceptibility of which to degradation by endogenous nucleases can be adjusted by chemical modification of the bases and the backbone structure.

In another embodiment, the present invention discloses adjuvants suitable for the method of the present invention. In a preferred embodiment, adjuvants are used which provide a depot effect for the allergens to be administered and which elicit Th2-type immune responses. Although adjuvants eliciting primarily a Th2-type immune response are potentially interfering with the induction of Tregs, in the presence of inhibitors of Il-4- and IL-13-mediated pathways such adjuvants are better suited for the therapeutic aims of the present invention than those eliciting primarily a Th1-type immune response. Preferred adjuvants eliciting Th2-type immune responses include but are not limited to aluminum salts, Montanide emulsions (squalene-based water-in-oil emulsions) and polyphosphazenes. Adjuvants eliciting a combined Th1- and Th2-type immune response may also be used for the method of the present invention if such adjuvants provide advantageous properties for the composition comprising the allergen(s), an adjuvant, inhibitors of IL-4- and IL-13-mediated pathways, and a matrix mediating prolonged delivery of such inhibitors. Adjuvants eliciting a combined Th1-type and Th2-type immune response include but are not limited to squalene-based oil-in-water emulsions, granulocyte-macrophage colony stimulating factor (GM-CSF), and adjuvants based on inulin and virosomes.

In another embodiment, the present invention discloses vaccine compositions which are useful for the method of the present invention. In one specific embodiment, the vaccine composition comprises a biodegradable thermogelling polymer solution containing one or more soluble inhibitors of IL-4- and IL-13-mediated pathways. Upon injection this fluid formulation then spontaneously gels as the temperature of the formulation rises to body temperature. Thereby, the injected gel forms a non-flowing depot from which one or more soluble inhibitors of IL-4- and IL-13-mediated pathway are released slowly and continuously for several days. The quantity of inhibitors of IL-4- and IL-13-mediated pathways in the composition is balanced in a way that upon gellation of the polymer composite at body temperature the amount of released inhibitors is sufficient for the therapeutic aims of the method of the present invention. For prolonged allergen presentation at the site of the gelled polymer composite, one or more allergens or an allergen extract are adsorbed onto aluminum salts or emulsified to form a squalene-based water-in-oil emulsion (Montanide emulsion), and injected in close proximity of the gelled polymer composite.

In another specific embodiment, the vaccine composition comprises a biodegradable thermogelling polymer solution containing one or more soluble inhibitors of IL-4- and IL-13-mediated pathway and one or more allergens (or an allergen extract) either adsorbed onto aluminum salts, or emulsified to form a squalene-based water-in-oil emulsion (Montanide emulsion), or premixed with a water-soluble polyphosphazenes polymer adjuvant. The quantity of each component in the composition is balanced in a way that a) upon injection into the body the biodegradable thermogelling polymer forms a non-flowing gel in which the other components are embedded, and b) upon gellation of the polymer composite at body temperature the amount of released components is sufficient for the therapeutic aims of the method of the present invention.

In still another specific embodiment, the vaccine composition comprises a biodegradable thermogelling polymer solution containing one or more allergens (or an allergen extract), one or more soluble adjuvants (such as GM-CSF), and one or more soluble inhibitors of IL-4- and IL-13-mediated pathways. The quantity of each component in the composition is balanced in a way that a) upon injection into the body the biodegradable thermogelling polymer forms a non-flowing gel in which the other components are embedded, and b) upon gellation of the polymer composite at body temperature the amount of released components is sufficient for the therapeutic aims of the method of the present invention.

In another embodiment, the present invention discloses methods for incorporating vaccine compositions into pharmaceutical compositions suitable for administration.

In yet another embodiment, the present invention discloses therapeutic methods for the induction of allergen tolerance using the vaccine compositions of the present invention.

Specific preferred embodiments of the present invention will become evident from the following more detailed description and the claims.

### DETAILED DESCRIPTION OF THE INVENTION

Based on current knowledge, allergen tolerance is mediated by peripherally induced regulatory T cells as evidenced by a deficit of allergen-specific IL-10 producing T cells in the peripheral blood of allergic patients (for reviews, see Schmidt-Weber et al., 2005; Schmidt-Weber et al., 2006). The main principle of specific immunotherapy in atopic patients is to increase the number of Tregs capable of controlling allergen-specific effector T cells. The expression of FOXP3 transcription factor is considered a key factor for the induction of regulatory T cells. We and others have shown that TCR receptor triggering is essential to induce FOXP3 expression and suppressive T cells in humans (Schubert et al., 2001; Mantel et al., 2006). We demonstrated that GATA3 induced by IL-4 inhibits the expression of FOXP3, which is a requirement for inducible Treg (iTreg) differentiation (Mantel et al., 2007). We have demonstrated this functional antagonism in vivo and in vitro and have established that the mechanistic basis for this is IL-4-dependent binding of GATA3 to the FOXP3 promoter with subsequent inhibition of FOXP3 transcription. As a consequence this antagonism torpedoes induction of Tregs and tolerance in conditions that are associated with excessive IL-4 production.

Under in vitro conditions, the antigen and the absence of Th1- or Th2-driving factors have been demonstrated to be essential for the induction of Tregs. Based on this observation it has been postulated that the generation of Tregs represents a default pathway that requires T-cell receptor activation as for any other T-cell differentiation, but the absence of decision signals for effector T cells. It is known, however, that IL-4 is released locally upon allergen injection due to the activation of Th2 memory T cells and IL-4 will also be released during the vaccination process. Therefore, in the present invention methods have been designed for coincident in vivo blockade of IL-4-mediated pathways along with allergen stimulation of T cells to augment the induction of clinical tolerance to allergen.

Since IL-13 is also known to be associated with the induction of the IgE isotype switch, secretion of IgE by B lymphocytes, and enhancement of IgE-mediated responses, methods of the present invention include also coincident in vivo blockade of IL-13-mediated pathways along with allergen stimulation of T cells. Although T cells lack an IL-13 binding receptor component and do not respond to IL-13, several studies indicate that IL-13 blockade could further augment the induction of clinical tolerance to allergen.

Most important, the present invention discloses methods for a prolonged inhibition of IL-4- and IL-13-mediated effects along with a prolonged presentation of the allergens. Prolongation of the process is essential since T cell differentiation and thus the development of immunologic memory requires the engagement of the T cell receptor (TCR) over 12-48 h and long lasting memory may even require repetitive exposure. Accordingly, inhibition of IL-4- and IL-13-mediated effects needs to be in effect at least for the same period of time, most likely as long as allergens are released from the injected allergen-adjuvant complex. Otherwise, released allergen molecules may induce Th2-driving factors, thereby inhibiting the induction of Tregs. The present invention discloses methods for a steady release of inhibitors of IL-4- and IL-13-mediated effects from a depot at the site of allergen presentation.

The present invention discloses also methods for establishing high local concentrations of inhibitors of IL-4- and IL-13-mediated effects at the site of allergen presentation which is as important as a prolonged presence of inhibitors. Since IL-4 can be shuttled in an antigen-specific manner in the immunological synapse that is formed between the antigen presenting cells and the T cells, effective inhibition of IL-4-mediated effects in the micro-environment of the immunological synapse requires a high local inhibitor concentration. Although T cells do not express IL-13Rα1, type II receptor complexes mediating IL-13-induced effects are expressed on both hematopoietic cells and non-hematopoietic cells in close proximity of the immunological synapse that is formed between the antigen presenting cells and the T cells. Therefore, effective inhibition of IL-13-mediated effects can be assumed to require also a high local inhibitor concentration.

High local concentrations of inhibitors, however, are extremely difficult to achieve via systemic administration and high doses of therapeutics are frequently associated with increased adverse effects. For example, the IL-4 mutein IL-4/Y124D (substitution of tyrosine with aspartic acid at position 124) binds to IL-4Rα with the same affinity as the wild-type IL-4, but has only 0.5% of the agonistic activity (Letzelter et al., 1998). This IL-4 mutein is a potent inhibitor of IL-4-mediated effects, but in vivo experiments have demonstrated that IL-4/Y124D exhibits acute toxicity upon systemic application similar to that of wild-type IL-4 in monkeys. The cellular events associated with both wild-type IL-4- and IL-4/Y124D-mediated toxicity include upregulation of VCAM-1 (vascular cell adhesion molecule 1), upregulation of MCP-1 (monocyte chemotactic protein 1) in serum, increases in circulating monocytes together with a concomitant decrease in circulating lymphocytes, and an increase in hematocrit (WO 97/47744). Similar cellular trafficking upon systemic application has been observed in clinical trials using IL-4 in humans (Wong et al., 1992). These results suggest that these toxicities of IL-4/Y124D are due to agonist activities mediated through cells other than T cells. Based on the known effects of Il-4 on endothelial cells, the observed toxicities of IL-4/Y124D point to interactions of this IL-4 mutein with a novel IL-4 receptor complex expressed on endothelial cells (WO 97/47744). This example demonstrates the necessity for restricting high local concentrations of inhibitors of IL-4- and IL-13-mediated effects mainly to the site of allergen presentation.

The method of the present invention solves this problem by disclosing procedures which utilize inhibitors of IL-4- and IL-13-mediated effects with a relatively short half-life. Such inhibitors are locally active upon their release from a depot at the site of allergen presentation and are quickly removed from circulation upon diffusion and transport away from the site of allergen presentation. For example, U.S. Pat. No. 6,028,176 and U.S. Pat. No. 6,313,272 disclose an IL-4 mutein with potent antagonistic activity but a relatively short plasma half-life in vivo of approximately 3 to 6 hours.

In consequence, the present invention discloses methods that provide a high local concentration of inhibitors for a prolonged period of time at the site of allergen presentation associated with a limited radius of inhibitory activity away from the site of allergen presentation due to rapid clearance. As a result, potential adverse effects mediated by the inhibitors are reduced significantly.

### I. Inhibitors of Decision Signals for T-Cell Differentiation

For the present invention several principles for local inhibition of the effects of IL-4 and IL-13 are suitable (for a review, see Long, 2009). Preferred principles include but are not limited to the application of antagonistic cytokine derivatives, soluble cytokine receptor constructs, monoclonal antibodies, fragments thereof or other proteinaceous constructs with specificity for IL-4 and Il-13 or their respective receptors, and anti-cytokine and anti-cytokine receptor aptamers. Any compound that functions as an IL-4, IL-13 or IL-4/IL-13 antagonist and is suitable for administration in accordance with the method of the present invention may be employed. Antagonists do not need to completely abolish IL-4- or IL-13-induced biological activity to be useful. Rather, a given antagonist may reduce a biological activity of IL-4 and/or IL-13. Combinations of two or more antagonists may be employed in methods and compositions of the present invention.

Most preferred inhibitors of IL-4 and IL-13 are those which a) provide a small to moderate molecular size and high solubility in aqueous environments for fast diffusion into the immunological synapse, b) can be embedded in substantial quantities in matrices suitable for continuous release of the inhibitor over a prolonged period of time, c) are releasable from a depot-forming matrix in sufficient quantities to provide high local concentrations at the site of allergen presentation, and d) have a relatively short plasma half-life to reduce potential adverse effects mediated by interaction of the inhibitor with targets away from the site of allergen presentation.

The vast majority of the anti-IL-4 and anti-IL-13 principles which are useful for the therapeutic aims of this invention, have been published and evaluated in animal models as well as in clinical trials for the treatment of asthma (for a review, see Long, 2009). However, application of these inhibitors according to the method of the present invention has not been disclosed.

Many inhibitors of IL-4- and IL-13-mediated pathways including antagonistic cytokine derivatives, soluble cytokine receptor constructs, anti-cytokine and anti-cytokine receptor antibodies have been developed to interrupt or to reduce Th2 dependent allergic inflammation in patients with asthma. The therapeutic aim is a shift of the patient's immune response towards a cytotoxic T-cell-mediated immune response, e.g., characterized by a Th1-type immune phenotype. In contrast, the therapeutic aim of the present invention is not a shift from a Th2-type immune phenotype to a Th1-type immune phenotype, but the induction of Tregs in a local micro-environment that is free of Th1- or Th2-driving factors despite the presence of allergens and adjuvants eliciting a Th2-type immune response.

In addition to applications for treatment of asthma, patent WO 2009/081 201 A2 discloses clinical applications of an IL-4Rα inhibitor for use a) in allergy therapy to suppress IgE synthesis (including for example atopic dermatitis and food allergy), b) for transplantation therapy to prevent transplant rejection, and c) for suppression of delayed-type hypersensitivity or contact hypersensitivity reactions. However, details of the suggested clinical applications are not disclosed.

In general, the rationale in published applications of IL-4 inhibition is a shift of the patient's Th2-type immune response towards a Th1-type immune response. For example, the application of inhibitors of IL-4-mediated pathways for the treatment of cancer is based on the assumption that it is sometimes beneficial for the patient to promote a Th1-type immune response (WO 02/04009 A2). Furthermore, U.S. Pat. No. 2011/0002013 A1 discloses that IL-4 antagonists find use as adjuvants to allergy immunotherapy and as vaccine adjuvants, especially when directing the immune response towards a Th1 response would be beneficial in treating or preventing the disease in question. Again, details of the suggested application of IL-4 antagonists as adjuvants to allergy immunotherapy are not disclosed.

### II. Inhibition of Decision Signals for T-Cell Differentiation by Interleukin Variants

In one embodiment of the invention, interleukin variants capable of antagonizing the activity of IL-4 and IL-13 are used as inhibitors. Any interleukin variant that functions as an IL-4, IL-13 or IL-4/IL-13 antagonist and is suitable for administration in accordance with the method of the present invention may be employed. Interleukin-based antagonists do not need to completely abolish IL-4- or IL-13-induced biological activity to be useful. Rather, a given antagonist may reduce a biological activity of IL-4 and/or IL-13.

Antagonistic IL-4 mutants have been described (Kruse et al., 1992; Muller et al., 1994; U.S. Pat. No. 6,028,176; U.S. Pat. No. 5,723,118). Within the four helix bundle of human IL-4, tyrosine (Y124) on the D helix forms an important contact with the common γ-chain (γ_{c}). When Y124 is mutated to aspartic acid (Y124D), the resulting molecule binds to IL-4Rα with the same affinity as the wild-type IL-4, but has only 0.5% of the agonistic activity (Letzelter et al., 1998). Similarly, the arginine at position 121 of human IL-4 interacts with IL-13Rα1. Mutations of R121 generates mutants that can bind to IL-4Rα, but are antagonists of both IL-4- and IL-13-induced responses (Kruse et al., 1993). However, other or additional mutations of IL-4 may also be considered for generating IL-4 variants suitable for the method of the present invention. For example, by mutation of serine at position 125 of IL-4 mutants have been generated that can bind to IL-4Rα, but are antagonists of both IL-4- and IL-13-induced responses (Kruse et al., 1993). Suitable for the method of the present invention are antagonistic interleukin-4 variants with single, double and triple mutations, single mutations including but not limited to amino acid positions R121, Y124, and S 125; double and triple mutations including but not limited to combinations of the above listed amino acid positions. Preferred are antagonistic IL-4 variants with a relatively short half-life to limit adverse effects distant from the site of allergen presentation.

The double mutant of human IL-4 (R121D/Y124D) has been shown to protect allergic cynomolgus monkeys from allergen-induced airways hyper-responsiveness when administered either subcutaneously or via nebulisation. Furthermore, subcutaneous administration of this double mutant in monkeys for 6 weeks or more also reduced the cutaneous wheal response and circulating concentrations of allergen-specific IgE. The effect of the double mutant of IL-4 (R121D/Y124D) on late phase asthmatic response to allergen challenge in asthmatic patients has been evaluated recently in clinical studies (Wenzel et al., 2007). The studies demonstrate that dual inhibition of IL-4 and Il-13 can positively affect the course of late asthmatic response after experimental allergen challenge. Treatment with the double mutant was associated with few adverse events, whether administered by subcutaneous injection (up to 30 mg for up to 13 weeks) or by inhalation (up to 60 mg for up to 4 weeks) in participants with atopic asthma or atopic eczema. However, it should be stressed that application of this double mutant for allergy immunotherapy according to the method of the present invention has not been disclosed.

More than 10 years ago animal experiments have demonstrated that inhibition of the IL-4/Il-13 receptor system can completely abrogate humoral immune response to allergen and development of allergic symptoms in vivo (Grunewald et al., 1999). Treatment of BALB/c mice with the murine IL-4 mutant Q116D/Y119D (the murine equivalent of the human IL-4 double mutant R121D/Y124D) before and after immunization with ovalbumin (OVA) completely inhibited synthesis of OVA-specific IgE and IgG1. In addition, the synthesis of specific IgG2a, IgG2b and IgG3 was suppressed, which may indicate the development of tolerance toward OVA. Immunization was performed by i.p. injection of 10 μg OVA in the presence of Alum as an adjuvant. At day 0, the murine IL-4 mutant was applied by i.p. injection 2 hours pre- and post-OVA-immunization as a 50 μg dose each. From day 1 to 8 treatment was continued with 30 μg mutant twice a day by i.p. injection. The authors concluded that the murine IL-4 mutant Q116D/Y119D inhibits antigen-specific humoral immune responses and allergic symptoms mediated either by IgE or IgG1 in vivo and, therefore, should be advantageous for therapy of atopic disorders and other Th2-dominated diseases. Although the results of this study are close to the method and the therapeutic aim of the present invention, the study of Grunewald et al. (1999) does not disclose how to provide a high local concentration of inhibitors for a prolonged period of time at the site of allergen presentation associated with a limited radius of inhibitory activity away from the site of allergen presentation due to rapid clearance. Grunewald et al. (1999) report the apparent induction of tolerance to the OVA allergen by starting the application of the murine IL-4 mutant Q116D/Y119D before immunization with OVA, which is not comparable to immunotherapy of patients with an existing allergy. For the induction of tolerance to allergens in individuals with a Th2-type immune phenotype only the method of the present inventions provides appropriate means to ensure a sufficient induction of Tregs in a local micro-environment that is free of Th1- or Th2-driving factors despite the presence of allergens.

IL-13 mutants useful for the method of the present invention include but are not limited to the IL-13 mutant E13K which is a powerful antagonist of human IL-13 and capable of partially inhibiting the responses of human IL-4 as well (Kioi et al., 2004). Glutamate at position 13 in IL-13 associates with IL-4Rα and a mutation to lysine decreases its binding to IL-4Rα. Although IL-13E13K prevents binding of IL-4Rα to IL-13Rα1, full inhibition of IL-4 responses cannot be achieved due to the availability of the alternative IL-4R complex, IL-4Rα:γ_{c}.

In a preferred embodiment the human IL-4 double mutant R121D/Y124D is used for inhibition of IL-4- and IL-13-mediated pathways in accordance with the method of the present invention. The human IL-4 double mutant R121D/Y124D is able to antagonize the activity of IL-4 as well as that of IL-13 (Grunewald et al., 1998; Tony et al., 1994).

### III. Inhibition of Decision Signals for T-Cell Differentiation by Soluble Cytokine Receptor Constructs

In one embodiment of the invention, soluble cytokine receptor constructs are used for the method of the present invention to inhibit IL-4- and IL-13-mediated pathways. Preferred constructs include but are not limited to the soluble extracellular domain of IL-4Rα, variants, fragments and derivatives thereof that retain the ability to bind IL-4. Human as well as murine IL-4 receptors and their nucleotide sequences have been described (U.S. Pat. No. 5,599,905; Idzerda et al., 1990 (human IL-4 receptor); Mosley et al., 1989 (murine IL-4 receptor)). The encoded human IL-4 receptor comprises an N-terminal signal peptide, followed by an extracellular domain, a transmembrane region corresponding to amino acids 208 through 231, and a cytoplasmic domain corresponding to amino acids 232 through 800. IL-4 receptor polypeptides arising from alternative mRNA constructs, e.g. which can be attributed to different mRNA splicing events following transcription, and which yield polypeptides capable of binding IL-4, are among the IL-4 receptor polypeptides disclosed herein. IL-4 receptor variants include those having amino acid or nucleotide acid sequences that vary from a native sequence by one or more substitutions, deletions, or additions, but retain a biological activity of the IL-4 receptor protein. The present invention also includes IL-4 receptor proteins with or without associated native-pattern glycosylation. The glycosylation pattern may vary according to the type of host cells in which the protein is produced. Another option is inactivation of N-glycosylation sites by site-directed mutagenesis. Derivatives of the IL-4 receptor protein include those exhibiting a different structure as compared to the native protein, having derivatized amino acids (e.g., by cross-linking reagents, or polyethylene glycol).

In another embodiment, the soluble extracellular domain of IL-4Rα, variants, fragments and derivatives thereof that retain the ability to bind IL-4, are conjugated chemically or fused to the N-terminus or C-terminus of other proteins or polypeptides. Preferred IL-4 receptor fusion proteins or polypeptides include but are not limited to peptides facilitating purification (e.g., His-tag) or identification (e.g., Flag peptide), and proteins or peptides that have the property of promoting oligomerization (e.g., leucine zippers). In a preferred specific embodiment, the soluble extracellular domain of IL-4Rα is fused to the Fc portion or selected constant domains of a human immunoglobulin of any isotype, or a fragment thereof capable of dimerization. If fusion proteins are made with both heavy and light chains of an antibody, it is possible to form an oligomer with as many as four extracellular regions of the IL-4 receptor. Preferred constructs include also hetero-oligomers in which both the soluble extracellular domain of IL-4Rα and the soluble extracellular domain of IL-13Rα1 are fused to a human protein or polypeptide capable of oligomerization.

Among cytokine receptor-based inhibitors suitable for the method of the present invention heterodimers containing both the soluble extracellular domain of IL-4Rα and the soluble extracellular domain of IL-13Rα1 are most preferred. The solubilized IL-4 receptor fragment consisting of the extracellular portion of the human IL-4R-α chain (shIL-4R) that competitively inhibits the binding of IL-4 to its receptor, appeared promising in early human studies (Borish et al., 2001). However, a subsequent large scale clinical trial indicated that this reagent had no clinical efficacy in asthma. This poor response could be a consequence of complex formation of shIL-4R with IL-13Rα1 on the cell surface, thereby stabilizing the weak interaction of IL-13 with IL-13Rα1 leading to enhanced IL-13 responses. In order to inhibit both IL-4- and IL-13-mediated responses, a soluble hetero-dimeric receptor construct has been generated which contains the extracellular domains of IL-4Rα and IL-13Rα1, both fused to the Fc portion of human IgG1 (Economides et al., 2003). This heterodimeric construct binds IL-4 and IL-13 with high affinity. IL-13 binds to non-complexed IL-13Rα1 with low affinity, but complexes of IL-13Rα1 and IL-4Rα form a high affinity binding site for IL-13 and IL-4 binds even to non-complexed IL-4Rα with high affinity (Andrews et al., 2006). Application of a heterodimeric construct providing binding sites for IL-13 and IL-4 for allergy immunotherapy according to the method of the present invention has not been disclosed.

### IV. Inhibition of Decision Signals for T-Cell Differentiation by Antibodies

In another embodiment, antibodies that function as IL-4 or IL-13 antagonists are employed for the method of the present invention. The antibodies preferably are monoclonal antibodies or antigen-binding fragments thereof. Advantageously, humanized or chimeric monoclonal antibodies are employed. Most preferred are human monoclonal antibodies. Examples of suitable antibodies are those that interact with the binding of IL-4 or IL-13 to an IL-4 receptor or Il-13 receptor, respectively. Such antibodies, referred to herein as blocking antibodies, may be raised against either the cytokines IL-4 and IL-13 or the cytokine receptors IL-4Rα and IL-13Rα1, and screened in conventional assays for the ability to interfere with binding of IL-4 and IL-13 with their respective receptor subunits. Antigen-binding fragments of such antibodies including Fab and F(ab′)₂ or artificial antibody constructs such as scFv or other proteinaceous constructs such as anticalins are also suitable for the method of the present invention. Additional embodiments include chimeric antibodies and antigen-binding fragments thereof, e.g., humanized versions of murine monoclonal antibodies, and human monoclonal antibodies as well as antigen-binding fragments thereof.

In a preferred embodiment, combinations of two or more humanized or human monoclonal antibodies with specificity for IL-4 and IL-13 which are capable of blocking the biologic activity of both cytokines, are used for the method of the present invention. In accordance with the method of the present invention, the biologic activity of both cytokines needs to be blocked to create a local micro-environment at the site of allergen presentation that is free of Th1- or Th2-driving factors.

Suitable monoclonal antibodies capable of blocking the biologic activity of IL-4 and IL-13 have been reviewed recently (Holgate et al., 2008; Long, 2009). For example, the anti-IL-4 humanized monoclonal antibody pascolizumab has been shown to effectively block IL-4 responses in vitro (Hart et al., 2002). Although this antibody yielded unimpressive results in the treatment of asthma, it may be a promising candidate for the method of the present invention. Binding of IL-13 to IL-4Rα can be blocked effectively with the fully humanized monoclonal IgG1 antibody IMA-638 and the interaction of IL-13 with IL-13Rα1 can be inhibited with the fully humanized monoclonal IgG1 antibody IMA-026 (Gauvreau et al., 2010). Both antibodies have shown efficacy in the treatment of asthma in cynomolgus monkeys, but only IMA-638 was able to reduce both early and late phase asthmatic responses in a clinical Phase II study (Gauvreau et al., 2010). Application of such antibodies, fragments and artificial constructs thereof, as well as other anti-cytokine proteinaceous constructs for allergy immunotherapy according to the method of the present invention has not been disclosed.

In another embodiment of the invention, antibodies capable of inactivating cytokine receptors are used to inhibit IL-4- and IL-13-mediated pathways. Preferred are humanized or human monoclonal antibodies with specificity for IL-4Rα which are capable of blocking the biologic activity of both IL-4 and IL-13. For example, the fully human monoclonal IgG2 antibody AMG317 binds with high affinity (IQ=1.8×10⁻¹⁰ M) to IL-4Rα and potently blocks the biologic activities of both IL-4 and IL-13 (Corren et al., 2010). Furthermore, AMG317 has been shown to have important effects on inflammation in vitro. Although in a Phase II study in patients with asthma AMG317 did not demonstrate clinical efficacy across the overall group of patients (Corren et al., 2010), this antibody provides preferred properties for the method of the present invention. However, it should be stressed that application of this antibody and other anti-cytokine receptor antibodies, fragments and artificial constructs thereof, as well as other anti-cytokine receptor proteinaceous constructs for allergy immunotherapy according to the method of the present invention has not been disclosed.

### V. Inhibition of Decision Signals for T-Cell Differentiation by Aptamers

In another embodiment of the invention, aptamers are used to inhibit IL-4- and IL-13-mediated pathways. Aptamers are nucleic acid binding species generated by iterative rounds of in vitro selection (for reviews, see Famulok et al., 1999; Yan et al., 2005). Typically, selected aptamers bind very tightly (often in the low nanomolar range) and specifically to their targets. Aptamers can also discriminate between closely related protein targets. Aptamers have several key features that make them particularly well suited as reagents for the method of the present invention. First, aptamers are relatively small and can readily access sites which are difficult to target with large molecules such as the immunological synapse that is formed between the antigen presenting cells and the T cells. Another advantage for the present invention is the possibility to engineer the stability of aptamers towards degrading nucleases, thereby determining the period of their inhibitory activity. Unmodified RNA- and DNA-based aptamers can begin degrading in serum within minutes, whereas aptamers containing modified bases and phosphorothioate linkages display a significantly increased stability towards degrading nucleases. For example, 2′-aminopyrimidine-modified RNA has been shown to remain stable for days. Furthermore, modifications can also be applied to the ends of aptamers to confer greater stability. For example, a 3′-3′ linkage can be added to prevent 3′-exonuclease degradation. Stable aptamers can be generated by the Spiegelmer-technology (for a review, see Famulok et al., 1999) or by utilizing peptide nucleic acids (EP 1 785 490 B1). Peptide nucleic acids (PNA) represents a class of nucleic acid analogues where the charged deoxyribose-phosphodiester backbone has been replaced by a peptide strand comprising N-(2-aminoethyl)glycine monomers with the nucleobases adenine, thymine, guanine and cytosine attached to their alpha-carbon. Due to its artificial character PNA provides complete resistance against nucleases and proteases.

In a preferred embodiment, combinations of two or more aptamers with specificity for IL-4 and IL-13 which are capable of blocking the biologic activity of both cytokines, are used for the method of the present invention.

In another preferred embodiment of the invention, aptamers capable of inactivating cytokine receptors are used to inhibit IL-4- and IL-13-mediated pathways. Preferred are aptamers with specificity for IL-4Rα which are capable of blocking the biologic activity of both IL-4 and IL-13.

In another preferred embodiment of the invention, aptamers are used which provide limited resistance against enzymatic degradation that is sufficient for effective inhibition of IL-4- and IL-13-mediated pathways at the site of allergen presentation and in its close proximity, but guarantees rapid degradation of the aptamer while diffusing away from this site. In a specific embodiment, the stability of an inhibitory aptamer is adjusted according to the requirement of the method of the present invention by modification of its structure including but not limited to the introduction of phosphorothioate linkages, modification of bases (e.g., by modification with 2′-aminopyrimidine), and addition of a 3′-3′ linkage to the end of the aptamer.

In another preferred embodiment of the invention, the physiological clearance of an inhibitory aptamer is adjusted according to the requirement of the method of the present invention. In cases of an extremely fast clearance, the plasma life-time of an inhibitory aptamer can be extended by attachment of lipids or high molecular weight compounds such as 40 kD polyethylene glycol moieties.

### VI. Matrices for Prolonged Delivery of Inhibitors of Decision Signals for T-Cell Differentiation

In a preferred embodiment, matrices are used for local delivery of inhibitors of IL-4- and IL-13-mediated pathways that a) can serve as depot for substantial quantities of the inhibitor or inhibitor combinations, b) allow the release of sufficient quantities of the inhibitor or inhibitor combinations for efficient local inhibition of IL-4- and IL-13-mediated pathways over a prolonged period of time (optimally for a few days), c) are biodegradable, and d) are chemically and physically compatible with the adjuvant and allergen (s) used for the induction of tolerance according to the method of the present invention.

In one embodiment of the invention, biodegradable polymers are used for controlled delivery of inhibitors of IL-4- and IL-13-mediated pathways. Preferred biodegradable polymers approved by FDA and used in a clinical trial, include but are not limited to poly(D,L-lactic acid), poly(lactic-co-glycolic acid) (PLGA), and copolymers of L-lactide and D,L-lactide. An important characteristic of such polymers is their ability to be applied locally which allows intralesional concentrations of the inhibitors to be sustained while systemic deleterious side effects are minimized. All FDA approved polymers have been studied extensively for their biocompatibility, toxicology, and degradation kinetics. Furthermore, these polymers have been shown to release embedded therapeutics for several hours up to 40 weeks in vitro and are effective for several weeks in vivo. For example, PLGA microspheres containing the anti-VEGF RNA aptamer EYE001 have been demonstrated to deliver the aptamer in a sustained manner with an average rate of 2 μg per day over a period of 20 days (Carrasquillo et al., 2003).

In a more preferred embodiment, injectable in situ-forming gel systems which are biodegradable, are used for controlled delivery of inhibitors of IL-4- and IL-13-mediated pathways. Preferred in situ-forming gel systems (hydrogels) undergo a sol-gel-sol transition, which is free flowing sol at room temperature and a non-flowing gel at body temperature. Compared to other biodegradable polymers, the injectable thermogelling polymers possess several advantages including easy preparation, high encapsulation efficiency of bioactive molecules such as inhibitors of IL-4- and IL-13-mediated pathways, and free of harmful organic solvents in the formulation process (Qiao et al. 2005).

In one specific embodiment, biodegradable thermogelling block polymers are used which are based on monomethoxy poly(ethylene glycol) (MPEG) including but not limited to a) diblock copolymers consisting of MPEG and poly(ε-caprolactone) (PCL) (Hyun et al., 2007), b) MPEG-b-(PCL-ran-PLLA) diblock copolymers (Kang et al., 2010), and c) diblock copolymers consisting of MPEG and PLGA (Peng et al., 2010). MPEG copolymers containing PCL provide the advantage that they do not create an acidic environment upon biodegradation in contrast to MPEG copolymers containing PLLA and PLGA (Hyun et al., 2007).

In another specific embodiment, biodegradable thermogelling triblock polymers are used including but not limited to a) PLGA-PEG-PLGA (Qiao et al., 2005), b) PEG-PLGA-PEG (Zhang et al., 2006), and c) PEG-PCL-PEG (PECE) (Gong et al., 2009 a). Various biodegradable thermogelling triblock polymers made up of PLGA and PEG are disclosed in patent application WO 99/18142. At lower temperatures, hydrogen bonding between hydrophilic PEG segments of the copolymer chains and water molecules dominate in aqueous solutions, resulting in the dissolution of these copolymers in water. As the temperature increases, the hydrogen bonding becomes weaker, while hydrophobic forces of the hydrophobic segments such as PLGA segments are getting stronger, leading to sol-gel transition. PEG, PLGA and PCL are well-known FDA-approved biodegradable and biocompatible materials which have been widely used in the biomedical field.

In another specific embodiment, biodegradable thermo-gelling diblock and triblock copolymers are used which consist of polyethylene oxide (PEO) and a biodegradable polyester such as poly-L-lactic acid (PLLA) (Jeong et al., 1997). These block copolymers, however, are a free flowing sol at a higher temperature and form a gel at a lower temperature. For example, a 23% aqueous solution of PEO-PLLA-PEO (Mᵣ 5,000-2,040-5,000) is a sol at 45° C. and becomes a gel at 37° C. By changing the biodegradable block length, the sol-gel transition temperature can be manipulated, e.g., increasing the PLLA block length increases the aggregation tendency of a block copolymer in water, resulting in a steepening of the gel-sol transition curve slopes and the onset of gelation at lower concentrations. The sol-gel transition temperature is a function of concentration as well as composition of a block polymer.

In still another specific embodiment, Poloxamers (trade name Pluronics) are used. Poloxamers are nonionic triblock copolymers composed of a central hydrophobic chain of polypropylene oxide) (PPO) flanked by two hydrophilic chains of poly(ethylene oxide) (PEO) (Gilbert et al., 1987). Poloxamers exhibit also sol-gel transition behavior in aqueous solutions and have been used for sustained delivery of several therapeutic agents. However, Poloxamers are not biodegradable and can be accumulated in the body which may lead to toxic side effects. Thus, the application of Poloxamers in biomedical fields has been greatly restricted. In a recent study, Pluronic F127 (100-unit PEO chain surrounding one 65-unit PPO) has been used to form composite thermosensitive hydrogels with PECE (Gong et al., 2009-b). Based on the results of this study Pluronic F127/PECE composite hydrogels are biocompatible with low cell cytotoxicity and, therefore, may also be suitable for the method of the present invention.

The various biodegradable thermogelling polymers provide different stability characteristics. For example, Poloxamer triblock polymers provide excellent thermosensitivity, but due to weak hydrophobicity of the PPO block such copolymers form fast eroding gels which have been reported to persist in vivo a few hours at most. Exposure of Poloxamer gels to phosphate-buffered saline under in vitro conditions demonstrated gel erosion within 2 days (Hyun et al., 2007). Similar results were observed when the polymer solutions were subcutaneously injected into rats. Poloxamer gels could not be observed after 2 days (Hyun et al., 2007). Different results were obtained with MPEG-PCL gels. Under in vitro conditions MPEG-PCL gels maintained their structural integrity for more than 28 days and after subcutaneous injection into rats MPEG-PCL gels maintained their structural integrity longer than 30 days. The stability of MPEG-PCL gels, however, may also create problems since the rate of degradation of PCL in vivo is rather slow (2-3 years) compared to that of PLA, PGA or PLGA (for a review, see Sinha et al., 2004). Thus, after serving the function in delivering inhibitors of IL-4- and IL-13-mediated pathways in vivo, MPEG-PCL copolymers may remain in the body under physiological conditions for an uncertain period. Therefore, most preferred biodegradable thermogelling polymers for the method of the present invention are those which maintain their structural integrity for a few days but do not remain in the body for more than a week.

In a preferred embodiment of the present invention, biodegradable thermogelling polymers are used which allow to modify their degradation kinetics. For example, PLLA segments can be incorporated into the PCL segment of MPEG-PCL copolymers, since PLLA provides better accessibility of water to the ester bonds of PLLA which enhances the hydrolytic degradation of the copolymer (Kang et al., 2010). The resulting MPEG-b-(PCL-ran-PLLA) diblock copolymers offer a therapeutic window that is adjustable from a few weeks to a few months by varying the amount of PLLA in the PCL segment (Kang et al., 2010). In another example, the rate of PLGA-PEG-PLGA hydrogel erosion can be modified by altering the molar ratio of DL-lactide/glycolide in the PLGA segment. The DL-lactide moiety is more hydrophobic than the glycolide moiety. Therefore, by increasing the molar ratio of DL-lactide/glycolide in the PLGA segment of PLGA-PEG-PLGA triblock copolymers, more stable hydrogels are formed due to stronger hydrophobic interactions among the copolymer molecules (Qiao et al. 2005).

Several of the biodegradable thermogelling polymers have been analyzed for their ability to mediate sustained release of proteins. Although different proteins such as cytokine mutants, soluble cytokine receptor constructs and antibodies are likely to affect the release behavior of each copolymer in individual ways, characterization of the release of model proteins such as bovine serum albumin (BSA) provides important information. Using composite hydrogels containing different percentages of PECE and Pluronic F127, the in vitro release behavior of BSA proved to be dependent on the hydrogel composition, initial BSA loading amount, and hydrogel concentration (Gong et al., 2009-b). Sustained release of BSA above 15 days was achieved with a composite hydrogel containing 60% PECE and 40% Pluronic F127, loaded with 4 mg BSA in the presence of 30 wt % hydrogel. Using MPEG-PCL copolymers, sustained in vitro release of BSA proved to be above 20 days, and under in vivo conditions (after subcutaneous injection into rats) sustained release lasted for more than 30 days (Hyung et al., 2007).

While for most clinical applications a sustained release of therapeutic drugs from biodegradable thermogelling polymers over a period of several weeks is desirable, the method of the present invention does not require such an extended sustained release of inhibitors of IL-4- and IL-13-mediated pathways since the development of immunologic memory requires the engagement of the T cell receptor (TCR) for a period of only 1 to 2 days. Furthermore, an extended sustained release of inhibitors of IL-4- and IL-13-mediated pathways over a period of several weeks may create local adverse effects. Therefore, preferred are biodegradable thermogelling polymers which deliver inhibitors of IL-4- and IL-13-mediated pathways for a limited period only which does not exceed a week.

PLGA-PEG-PLGA triblock copolymers represent one example of hydrogels providing advantageous degradation and release kinetics for the method of the present invention. As demonstrated in a recent study, the release of insulin from PLGA-PEG-PLGA triblock copolymers lasted for approximately 4 days with an initial burst effect during the first day (Choi et al, 2003). The initial burst effect may be advantageous for complete inhibition of IL-4- and IL-13-mediated pathways in the initial phase of allergen presentation. However, the release kinetics may be modified by reducing the solubility of inhibitors of IL-4- and IL-13-mediated pathways via complexation with zink as shown for zink-insulin complexes (Choi et al, 2003).

Although Poloxamer gels are not biodegradable, Poloxamer 407 (trade name Pluronic F127) gels represent also an example of thermogelling polymers providing advantageous degradation and release kinetics for the method of the present invention. Although under in vitro conditions the release of BSA from Poloxamer 407 gels proved to be almost complete within 1 day, the sustained release of BSA under in vivo conditions (after subcutaneous injection into rats) lasted for 3 days (Hyung et al., 2007).

### VII. Adjuvants

The time of antigen exposure is extremely important since T cell differentiation and thus the development of immunologic memory requires the engagement of the T cell receptor (TCR) over 12-48 h and long lasting memory may even require repetitive exposure. Adjuvants such as oils and alum provide a depot effect that extends the lifetime of antigens and thus prolong antigen-stimulation of T cells. However, in addition to the depot effect currently available adjuvants trigger either Th1-type or Th2-type immune responses or both (for reviews, see Leroux-Roels, 2010; Nicholls et al., 2010). Based on the assumption that the generation of Tregs represents a default pathway which requires T-cell receptor activation but the absence of decision signals for effector T cells, currently available adjuvants are not optimal for the purpose of the present invention. For the method of the present invention adjuvants are needed that promote allergen uptake and prolonged stimulation of T cells without providing decision signals, thereby inducing Treg expansion. Unfortunately, adjuvants providing all of the desired properties are not available.

Liposomes, synthetic nanospheres consisting of lipid layers, represent a classic adjuvant that is not active per se. Their main mode of action is through allergen presentation. Liposomes, can encapsulate allergens and act as allergen delivery vehicles. However, liposomes are not suitable for a slow release of allergens, which appears to be essential for a prolonged immune stimulation.

In a preferred embodiment of this invention, adjuvants eliciting Th2-type immune responses including but not limited to aluminum salts, Montanide emulsions (squalene-based water-in-oil emulsions) and polyphosphazenes, are used for the method of the present invention. Although adjuvants elicting primarily a Th2-type immune response are potentially interfering with the induction of Tregs, in the presence of inhibitors of Il-4- and IL-13-mediated pathways such adjuvants are better suited for the therapeutic aims of the present invention than those eliciting primarily a Th1-type immune response. In the presence of inhibitors of Il-4- and IL-13-mediated pathways the contra-productive activity of adjuvants eliciting Th2-type immune responses is significantly reduced or even abolished, whereas the activity of Th1-type promoting adjuvants cannot be influenced. As a result, Th1-type cytokines would interfere with the induction of Tregs.

In one preferred specific embodiment, aluminum-containing adjuvants, typically aluminum phosphate or aluminum hydroxide gels (generally referred to as alum) are used for the method of the present invention. One mechanism of action is due to its retention of immunogenic molecules at specific sites in the body, allowing for their slow release and consequently a prolonged immune response, the so-called depot effect. Alum also appears to exert its adjuvantic activity by keeping antigens in a particulate (rather than soluble) form, thereby enhancing phagocytosis of antigens by APCs. Dendritic cells (DC) are capable of taking up both soluble antigens and antigens adsorbed onto aluminum-containing adjuvants. Furthermore, it has become evident that alum is capable of directly activating immune cells. Alum can induce maturation of monocytes/macrophages into DC-like cells. This process is dependent on the activation of NLRP3 (NOD-like receptor family, pyrin domain containing 3), part of the inflammasome which causes production of pro-inflammatory cytokines such as IL-1β and IL-18. Primarily, alum elicits a Th2 response including IL-4 and IL-5 production as well as IgG1 and IgE production by B cells (for a review, see Nicolls et al., 2010).

Upon incubation of allergens with aluminum-containing adjuvants, the allergens are partially or completely adsorbed onto the adjuvant via electrostatic interactions and exchange between phosphate and hydroxyl groups. Several studies have demonstrated that adsorption onto aluminum-containing adjuvants prevents rapid enzymatic degradation of the allergens, but the adsorption process may also induce structural changes resulting in a decreased thermal stability (for a review, see Clapp et al., 2011). One approach to improving the thermal stability of vaccines is to use a combination of buffers to alter the surface chemistry of aluminum-containing adjuvants and to control the ionization state of the allergen's amino acid side chains. Another approach is to identify conditions that stabilizes the allergen(s) in the solution state and to use this as guidance on minimizing the extent of adsorption-induced thermal destabilization of the allergen(s). For practical purposes, however, stabilization procedures may be omitted since the importance of antigen (including allergen) adsorption to aluminum-containing adjuvants for the immunological response has been questioned by recent studies (for a review, see Clapp et al., 2011). For example, the immune response elicited in rabbits against hen egg white lysozyme was irrespective of the fraction of antigen adsorbed onto the aluminum-containing adjuvant (Chang et al., 2001). For maximizing the immune response, however, some degree of adsorption or at least the proximity of the allergen and the adjuvant appears to be important.

In another preferred specific embodiment, polyphosphazenes (water-soluble polymers) are used for the method of the present invention. Poly[di(carboxylatophenoxy)phosphazene] (PCPP) has been shown to enhance antibody responses while being negligibly reactogenic (Nicolls et al., 2010).

In still another specific embodiment, squalene-based water-in-oil emulsions (Montanide emulsions) are used for the method of the present invention. Montanide emulsions are similar to incomplete Freund's adjuvant, but they are biodegradable and therefore significantly less toxic and have been used in several clinical trials (Nicolls et al., 2010).

In another embodiment, adjuvants eliciting a combined Th1- and Th2-type immune response are used for the method of the present invention. Although adjuvants eliciting a combined Th1- and Th2-type immune response are likely to interfere to a certain extent with the induction of Tregs, such adjuvants may provide advantageous properties for the composition, which may be beneficial for the method of the present invention. Adjuvants eliciting a combined Th1-type and Th2-type immune response include but are not limited to squalene-based oil-in-water emulsions, granulocyte-macrophage colony stimulating factor (GM-CSF), and adjuvants based on inulin and virosomes (for a review, see Nicholls et al., 2010).

### VIII. Vaccine Compositions Comprising Inhibitors of IL-4- and IL-13-Mediated Pathways, a Matrix Mediating Prolonged Delivery of Such Inhibitors, Allergens and Adjuvants.

In order to achieve prolonged presentation of the allergen(s) along with a prolonged inhibition of IL-4- and IL-13-mediated effects at the site of allergen presentation, both the allergen(s) and inhibitor(s) are co-administered in a manner that limits diffusion of these components away from the site of allergen presentation. Useful diffusion limiting techniques for the method of the present invention include but are not limited to adsorption of components onto aluminum-containing materials, preparation of water-in-oil or oil-in-water emulsion with components, and incorporation of components into biodegradable polymers including biodegradable thermogelling polymers.

For some components it is useful to combine two or more of the diffusion limiting techniques to achieve the therapeutic aims of the present invention. One important aspect of the method of the present invention is the compatibility of the components in the therapeutic composition For example, adsorption of allergens and inhibitors of IL-4- and IL-13-mediated pathways onto aluminum-containing materials provides a prolonged presentation of the allergens together with a Th2-type adjuvant effect, but it is questionable whether the delayed release of the inhibitor(s) is sufficient for effective inhibition of IL-4- and IL-13-mediated pathways during allergen presentation. Incorporation of the inhibitors into a fast degrading polymer provides a better opportunity to achieve effective inhibition of IL-4- and IL-13-mediated pathways during allergen presentation. Therefore, a combination of both techniques is likely to guarantee a higher therapeutic efficacy than each of the techniques alone. Similar considerations apply to the use of water-in-oil or oil-in-water emulsion as diffusion limiting technique for inhibitors of IL-4- and IL-13-mediated pathways, since the release kinetics of inhibitors from such emulsions may not be sufficient for the requirements of the method of the present invention. However, biodegradable polymers have the potential to provide both prolonged allergen presentation and a sustained release of inhibitors of IL-4- and IL-13-mediated pathways sufficient for the requirements of the method of the present invention. A recent study has demonstrated that soluble adjuvants embedded together with antigens in biodegradable hydrogels are potent compositions for eliciting immune responses. Using diblock copolymers consisting of MPEG-PLGA for hydrogel-codelivery of hepatitis B surface antigen (HBsAg) and GM-SCF, it was possible to elicit high HBsAg-specific antibodies and T-helper cell responses even in a mouse strain that does not respond to current HBsAg vaccine because of its H-2 haplotype (Chou et al., 2010).

In one preferred embodiment of the present invention, the vaccine composition comprises a biodegradable thermogelling polymer containing one or more allergens or an allergen extract partially or completely adsorbed onto aluminum-containing adjuvants, and one or more soluble inhibitors of IL-4- and IL-13-mediated pathways. The composition is prepared by mixing all components at a temperature at which the biodegradable thermogelling polymer is a free flowing sol (e.g., at room temperature). The quantity of each component in the composition is balanced in a way that a) upon injection the biodegradable thermogelling polymer forms a non-flowing gel in which the other components are embedded, and b) upon gellation of the polymer composite at body temperature the amount of released components is sufficient for the therapeutic aims of the method of the present invention.

In another preferred embodiment of the present invention, polymer-embedded inhibitors of IL-4- and IL-13-mediated pathways and alum-adsorbed allergens (or allergen extract) are injected at the same location, but as separate preparations. After gellation of the polymer composite at the injection site, alum-adsorbed allergens (or allergen extract) are injected in close proximity of the gelled polymer composite. The polymer composite comprises a biodegradable thermogelling polymer and one or more soluble inhibitors of IL-4- and IL-13-mediated pathways, and is prepared by mixing all components at a temperature at which the biodegradable thermogelling polymer is a free flowing sol (e.g., at room temperature). The quantity of each component in the composition is balanced in a way that a) upon injection the biodegradable thermogelling polymer forms a non-flowing gel in which the other components are embedded, and b) upon gellation of the polymer composite at body temperature the amount of released components is sufficient for the therapeutic aims of the method of the present invention.

In another preferred embodiment of the present invention, the vaccine composition comprises a biodegradable thermogelling polymer containing one or more allergens or an allergen extract premixed with an adjuvant consisting of a squalene-based water-in-oil emulsion (Montanide emulsion), or a water-soluble polyphosphazenes polymer, and one or more soluble inhibitors of IL-4- and IL-13-mediated pathways. The composition is prepared by mixing all components at a temperature at which the biodegradable thermogelling polymer is a free flowing sol (e.g., at room temperature). The quantity of each component in the composition is balanced in a way that a) upon injection the biodegradable thermogelling polymer forms a non-flowing gel in which the other components are embedded, and b) upon gellation of the polymer composite at body temperature the amount of released components is sufficient for the therapeutic aims of the method of the present invention.

In another preferred embodiment of the present invention, polymer-embedded inhibitors of IL-4- and IL-13-mediated pathways, and allergens (or allergen extract) either embedded in a Montanide emulsion or mixed with a water-soluble polyphosphazenes polymer are injected at the same location, but as separate preparations. After gellation of the polymer composite at the injection site, the allergens (or allergen extract) either embedded in a Montanide emulsion or mixed with a water-soluble polyphosphazenes polymer are injected in close proximity of the gelled polymer composite. The polymer composite comprises a biodegradable thermogelling polymer and one or more soluble inhibitors of IL-4- and IL-13-mediated pathways, and is prepared by mixing all components at a temperature at which the biodegradable thermogelling polymer is a free flowing sol (e.g., at room temperature). The quantity of each component in the composition is balanced in a way that a) upon injection the biodegradable thermogelling polymer forms a non-flowing gel in which the other components are embedded, and b) upon gellation of the polymer composite at body temperature the amount of released components is sufficient for the therapeutic aims of the method of the present invention.

In still another preferred embodiment of the present invention, the vaccine composition comprises a biodegradable thermogelling polymer containing one or more allergens or an allergen extract, one or more soluble adjuvants eliciting a combined Th1- and Th2-type immune response (such as GM-CSF), and one or more soluble inhibitors of IL-4- and IL-13-mediated pathways. The composition is prepared by mixing all components at a temperature at which the biodegradable thermogelling polymer is a free flowing sol (e.g., at room temperature). The quantity of each component in the composition is balanced in a way that a) upon injection the biodegradable thermogelling polymer forms a non-flowing gel in which the other components are embedded, and b) upon gellation of the polymer composite at body temperature the amount of released components is sufficient for the therapeutic aims of the method of the present invention.

### IX. Therapeutic Methods

As used herein, a decrease or modification of the T cell response of an individual sensitive to a protein allergen is defined as diminution in symptoms to the protein allergen in the individual, as determined by standard clinical procedures (see e.g., Brit. Med. J. 1990; 302: 265). As referred to herein, a diminution in symptoms to an allergen includes any reduction in the allergic response of an individual to the allergen following a treatment regimen with a polypeptide as described herein. This diminution in symptoms may be determined subjectively (e.g., the patient feels more comfortable upon exposure to the allergen), or clinically, such as with a standard skin test.

For immunotherapy, the vaccine compositions of the present invention comprising one or more allergens, one or more adjuvants, one or more inhibitors of IL-4- and IL-13-mediated pathways, and a matrix mediating prolonged delivery of such inhibitors, are repeatedly administered at intervals as defined in various published immunotherapy protocols. The dose of the allergen or allergens in the vaccine compositions is gradually increased to an extent that is effective to reduce the allergic response of the individual to the allergen(s). In a particular embodiment, non-embedded inhibitors of IL-4- and IL-13-mediated pathways may be co-administered with the vaccine compositions to enhance the inhibitory effect at the site of administration. Preferred examples of routes of administration include but are not limited to intradermal and subcutaneous administrations. Dosage regimens may be adjusted to provide the optimum therapeutic response. For example, several divided doses may be administered daily.

In one preferred embodiment of the present invention, polymer-embedded inhibitors of IL-4- and IL-13-mediated pathways and alum-adsorbed allergens are applied for immunotherapy. First, a biodegradable thermogelling polymer solution containing one or more soluble inhibitors of IL-4- and IL-13-mediated pathways is injected subcutaneously. The quantity of the polymer-embedded inhibitors will depend on the release kinetics of the biodegradable thermogelling polymer and is adjusted to a level that guarantees the continuous release of therapeutically effective doses over a period of 3 to 5 days. In a second step, alum-adsorbed allergens are injected subcutaneously at the site of the gelled polymer composite. The quantity of alum-adsorbed allergens will vary according to factors such as the degree of sensitivity of the individual to the allergen(s), the age, sex, and weight of the individual, and the ability of the composition to induce tolerance toward the allergen(s) in the individual. In subsequent steps, polymer-embedded inhibitors are injected first, followed by the injection of alum-adsorbed allergens at the site of the gelled polymer composite. While the quantity of polymer-embedded inhibitors remains constant, the quantity of alum-adsorbed allergens is gradually increased to an extent that is effective to reduce the allergic response of the individual to the allergens. Both polymer-embedded inhibitors and alum-adsorbed allergens are repeatedly administered at intervals as defined in various published immunotherapy protocols until allergen tolerance is achieved.

The determination of a therapeutically effective dose is well within the capability of those skilled in the art. The therapeutically effective dose can be estimated initially in animal models, usually mice, rats, rabbits, dogs, pigs, or non-human primates. The animal model also can be used to determine the appropriate concentration range and route of administration. Such information can then be used to determine useful doses and routes for administration in humans.

### X. Pharmaceutical Formulations

In one embodiment, the vaccine compositions are incorporated into pharmaceutical compositions suitable for administration. Such compositions typically comprise the vaccine composition and a pharmaceutically acceptable carrier. As used herein, a ‘pharmaceutically acceptable carrier’ is intended to include any and all solvents, dispersion media, coatings, antibacterial and antifungal agents, isotonic systems, and the like, compatible with the components of the vaccine composition and pharmaceutical administration. The use of such media and agents for pharmaceutically active substances is well known in the art. Supplementary active compounds can also be incorporated into the composition.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents, antioxidants such as ascorbic acid or sodium bisulfite, chelating agents such as ethylenediaminetetraacetic acid, buffers such as acetates, citrates or phosphates and agents for the adjustment of toxicity such as sodium chloride or dextrose. The pH can be adjusted with acids or bases, such as hydrochloric acid or sodium hydroxide. In many cases, it will be preferable to include isotonic agents, for example, sugars, polyalcohols such as mannitol, sorbitol, sodium chloride in the composition. The composition should be fluid to the extent that easy syringability exists. The proper fluidity can be maintained, for example, by the use of a coating such as lecithin, by the maintenance of the required particle size in the case dispersion and by use of surfactants. The composition must be stable under the conditions of manufacture and storage and must be preserved against the contaminating action of microorganisms such as bacteria and fungi. Prevention of the action of microorganisms can be achieved by various antibacterial and antifungal agents such as parabens, chlorobutanol, phenol, ascorbic acid, thimoseral, and the like. In all cases, the composition must be sterile. Sterile injectable solutions can be prepared by filtered sterilization. The preparation can be enclosed in ampoules, disposable syringes or multiple dose vials made of glass or plastic. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof. For practical purposes it should be kept in mind that aluminum-adsorbed vaccines are frost sensitive and therefore not lyophilizable.

### BRIEF DESCRIPTION OF THE DRAWINGS

FIG. 1 shows a schematic representation of constructed expression vectors for different expression systems. 6 vectors were constructed for expression of murine IL-4 mutein QY in E. coli, 2 for expression in insect cells (TriEx+pACgp67), and 2 for expression in mammalia cells (pTriEx+pcDNA3.1). Test expression showed that the expression using the pETER-22b+ construct delivered the best protein yield per expression time, despite the necessity of refolding. 3 procaryotic vectors were constructed for expression of human IL-4 mutein RY (pTriEX, pET22b+, pETER-22b+).

FIG. 2 depicts the PAGE analysis of Ni-column purified and refolded mIL4 QY, expressed in pTriEx in Tuner (DE3). Reducing sample buffer conditions. From left to right: M=Protein size marker (PageRuler™ Plus, Fermentas), 1-8=Ni-NTA column elution fractions. The protein was expressed, but the yield was comparably low.

FIG. 3 shows the PAGE analysis of Ni-column purified and refolded mIL4 QY, expressed in pETER-22b+ and BL21(DE3). Coomassie stain, reducing sample buffer conditions. From left to right: M=Protein size marker (PageRuler™ Plus, Fermentas), I=purified inclusion bodies in 8 M urea, 1-8=fractions 11, 13, 15, 17, 19, 21, 23, and 25. The protein was expressed in comparably better yield than in the original TriEx construct.

FIG. 4 shows the PAGE analysis of cleavage of MBP-fusion protein of mIL4QY with pMalc2x in E. coli XL10Gold. Coomassie stain, reducing sample buffer conditions. From left to right: M=Protein size marker (PageRuler™ Plus, Fermentas), 1=MBP-mutein fusion protein with recombinant TEV protease (Mobitec, His-tagged) incubated for 1 hour at 30° C. 2=Cleavage reaction after purification by amylose matrix in batch process. 3=Cleavage reaction after purification by Ni-NTA matrix in batch process. Cleavage occurs, however seems not to be complete. Purification of the cleavage reaction by amylose and Ni-NTA suggests that either the cleaved mIL4QY binds nonspecifically to the matrix or the amount is not sufficient to be detected.

FIG. 5 shows the PAGE analysis of CBD-fusion protein expression of mIL4QY with pTYB11 in E. coli BL21(DE3). Coomassie stain, reducing sample buffer conditions. From left to right: M=Protein size marker (PageRuler™ Plus, Fermentas), 1=Supernatant of sonificated cells. 2=Insoluble protein of sonificated cells, dissolved in 8 M urea.

FIG. 6 shows a western blot analysis of cell lysates from different expression containing mIL4 QY, expressed in pAcgp67B in SF9, using anti-V5 antibody. Reducing sample buffer conditions. From left to right: M=Protein size marker (PageRuler™ Plus, Fermentas), 1=Cell lysate round 1, 1 μl virus stock, 2=Cell lysate round 1, 5 μl virus stock, 3=Cell lysate round 1, 25 μl virus stock, 4=Cell lysate round 2, 1 μl virus stock, 5=Cell lysate round 2, 5 μl virus stock, 6=Cell lysate round 2, 25 μl virus stock, 7=Cell lysate round 3, 1 μl virus stock, 8=Cell lysate round 3, 5 μl virus stock, 9=Cell lysate round 3, 25 μl virus stock. The protein could only be faintly detected in the supernatant (data not shown), most protein was detected in the lysates of the cells, especially with highest MOI of round 1 and 2. The pattern of the bands suggests that the signal peptide was only partially cleaved.

FIG. 7 shows the comparison of the refolding procedure according to Levine et al and refolding on Ni-matrix, using His-tag. PAGE gel, coomassie stain, reducing sample buffer conditions. From left to right: M=Protein size marker (PageRuler™ Plus, Fermentas), I1=Inclusion bodies according to Levine et al procedure. L=Soluble mIL4 protein at the end of the Levine et al purification scheme. P=Precipitated protein at the end of the Levine et al purification scheme. I2=Inclusion bodies according to Ni-matrix procedure. N=Soluble mIL4 protein at the end of the Ni-matrix purification scheme.

FIG. 8 shows the CD (circular dichroism) spectrum of refolded mIL4QY by Ni-matrix refolding procedure, in 10 mM MES pH 5.0. The spectrum is very similar to the published one of human IL-4. The alpha-helical structure is clearly visible. The spectrum hints at only minor amounts of unfolded protein, if at all.

FIG. 9 shows the Inhibition of IL-4 induced proliferation by mIL-4 QY mutein. The inhibition of murine B-cell proliferation, induced with 1 μg/ml anti-CD40 and 3 ng/ml murine IL-4, is dose-dependent on mutein concentration. Cell numbers are quantified by FACS sorting and anti-CD19 labeling of murine B-cells. Addition of different concentrations of mIL-4 QY mutein without IL-4 stimulation resulted in no significant change in B-cell numbers (data not shown).

FIG. 10 shows the determination of the gelation temperature of hydrogel solution. At different temperatures the gel is checked for gelation by tilting the test tube. A) lower temperature then gelation temperature. B) gelation temperature or higher.

FIG. 11 shows the release of mutein from 200 μl of 25% hydrogel in 1×PBS with 1.8 ml PBS overlay during incubation at 37° C. The concentration of mutein is determined by Sandwich ELISA in comparison to 100% sample (1 μg mutein in 2 ml PBS). The y-axis shows the amount of mutein released into buffer in percent. The x-axis shows the incubation time in hours.

### EXAMPLES

The following examples are intended to illustrate but not limit the present invention.

### Example 1

### Cloning, Expression and Purification of the Murine IL-4 Antagonist QY

A major obstacle for testing the effects of human antagonistic IL-4 mutants in vivo is the species specificity of IL-4. Human IL-4 does not bind to the mouse receptor and vice versa. Therefore, the murine IL-4 antagonist QY (Q116D/Y119D) in which the amino acids glutamine 116 and tyrosine 119 are mutated to aspartic acid, has been generated for the proof of concept. This murine IL-4 mutant is analogous to the R121D/Y124D double mutant of human Il-4, binds with high affinity to the murine IL-4Rα without inducing signal transduction, has no detectable activity upon proliferation or differentiation of murine cells, and an excess of the murine QY mutant has been shown to completely inhibit responses toward wild-type murine IL-4 (Grunewald et al., 1997). Furthermore, the murine QY mutant is also a complete inhibitor for murine IL-4 in the absence of γ (Andersson et al., 1997).

### Cloning of the Murine IL-4 Antagonist QY (Q116D/Y119D).

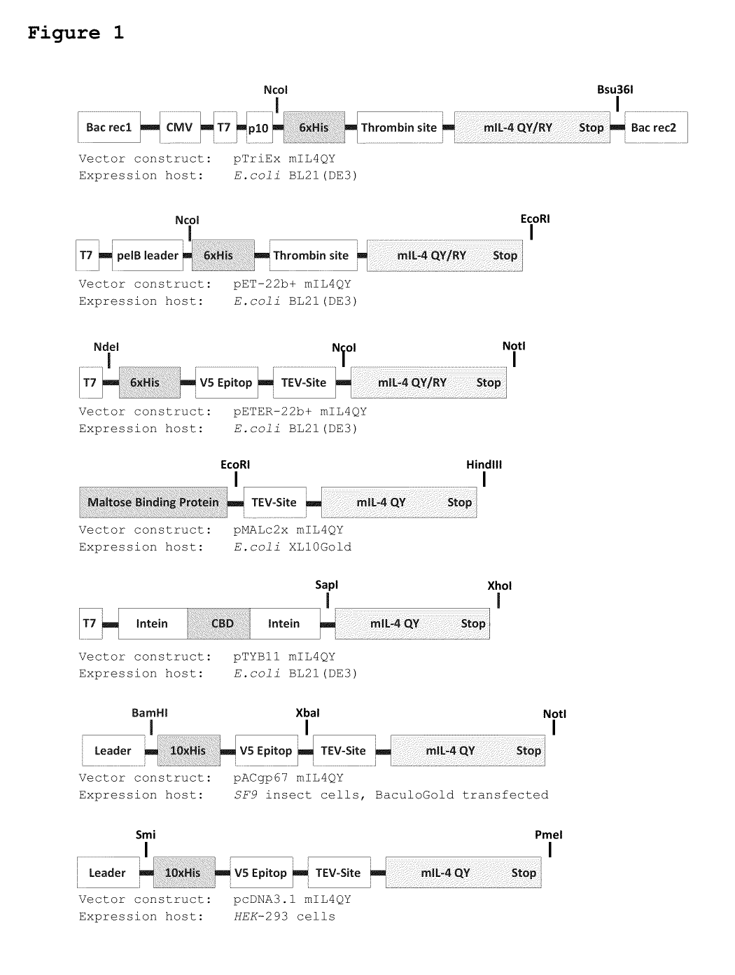

The sequence of the native mature murine IL4 protein is modified to include a thrombin cleavable N-terminal 6× His-tag and the QY (Q116D/Y119D) mutations (see SEQ ID 9). The protein sequence is translated into DNA using codon optimization for expression in prokaryotic E. coli cells (Puigbo et al, 2007; Grote et al, 2005). The sequence (SEQ ID 10) is synthesized and cloned into the E. coli expression vector pTriEx using NcoI and Bsu36I restriction sites (pTriEx mIL-4 QY). The mutein sequence is also re-cloned into the vectors pET-22b+(Novagen, prokaryotic expression in BL21(DE3), N-terminal pelB leader and 6× His Tag), pMAL-c2X (New Englang Biolabs, prokaryotic expression in XL10Gold, maltose binding protein fusion protein with TEV cleavage site), pTYB11 (New England Biolabs, prokaryotic expression in BL21(DE3), Fusion with self-cleaving Intein-Chitin-binding-domain), pETER-22b+(prokaryotic expression in BL21(DE3, modified pET-22b+ without pelB leader, with N-terminal 6× His- and V5-epitope and TEV cleavage site), pACgp67 (eukaryotic expression in BaculoGold transfected SF9 insect cells and pcDNA3.1 (eukaryotic expression in HEK-293, with 10× His- and V5-epitope and TEV cleavage site). A schematic representation of all vector constructs is given in FIG. 1. A comparison of protein expression of these constructs shows that the pETER-22b+ construct provide the best expression properties in terms of protein yield per expression time.

For the construction of the pET-22b+ construct The mutein sequence (SEQ ID 10) in pTriEX is amplified with primers 1 (IL4his-for, SEQ ID 5) and 3 (MOIL4mut-back, SEQ ID 11) using PCR with Pfu polymerase according to the manufacturer and annealing temperatures rising from initial 5 cycles at 50° C. to 60° C. and 30 cycles total. The resulting PCR fragment is gel-purified in 1% agarose (GeneJET Gel Extraction Kit, Fermentas) and transferred to a restriction enzyme double digest containing Nde I and Xho I (Fermentas). The restriction digest is again gel-purified and the fragment is ligated into Nde I and Xho I cut pET-22b (Novagen) expression vector. The ligation product is transformed into chemically competent E. coli BL21(DE3). The clone pET-22b+mIL4QY#4 is sequence verified and selected for expression studies.

For the construction of the pETER-22b+ construct the pET-22b+mIL4QY#4 is used as template for PCR using primers mIL4NcoI (SEQ ID 12) and J#90 (SEQ ID 13). The PCR is performed using Pfu polymerase according to the manufacturer and an annealing temperature of 58.5° C. and 30 cycles total. The resulting PCR fragment is gel-purified in 1% agarose (GeneJET Gel Extraction Kit, Fermentas) and transferred to a restriction enzyme double digest containing NcoI and Not I (Fermentas). The restriction digest is again gel-purified and the fragment is ligated into Nco I and Not I cut pETER-22b+ expression vector. The ligation product is transformed into chemically competent E. coli BL21(DE3). The clone pETER-22b+mIL4QY#8.1 is sequence verified and selected for expression studies.

### Expression.

The pTriEx mIL-4 QY construct is transformed into different E. coli strains (Origami™2(DE3)pLacI, Rosetta™(DE3)pLacI, Rosetta-gami B™(DE3)pLacI, BL21(DE3)pLysS, Tuner™(DE3)pLysI) containing the DE3 genetic element. The transformed bacteria are grown in 2xYT medium shaking at 220 rpm at 37° C. (Innova 4300 incubator, New Brunswick Scientific) containing the appropriate antibiotics and 1% glucose. At OD600 of 0.8 cells are induced with 1 mM IPTG and incubated at 37° C. with further shaking for 3 hours. The cells are centrifuged for 20 minutes at 11.000×g and 4° C. (Eppendorf 5804R centrifuge). The supernatant is discarded and the pellet resuspended in half volume (corresponding to expression culture volume) of lysis buffer (50 mM Tris, 5 mM sodium-EDTA, pH 8.0) with freshly added lysozyme (0.1 mg/ml). The pellet can be stored at −20° C. until starting of the refolding procedure.

E. coli BL21(DE3) (Novagen) containing the pETER-22b+mIL4QY#8.1 are grown in 500 ml 2xYT medium until OD600=0.8-1 at 37° C. with shaking (220 rpm). Expression is induced with addition of 1 mM IPTG and cells are incubated at 37° C. with further shaking for 3 hours. The cells are centrifuged for 30 minutes at 5.000×g and 4° C. (Eppendorf 5804R centrifuge). The supernatant is discarded and the pelleted cells are resuspended and combined in 50 ml medium. The cells are again centrifuged for 15 minutes at 5.000×g and 4° C. (Eppendorf 5804R centrifuge). The supernatant is discarded and the cell pellet is frozen at −20° C.

Other vector constructs are expressed according to the recommendations of the manufacturer. Schematic representations of these constructs are given in FIG. 1.

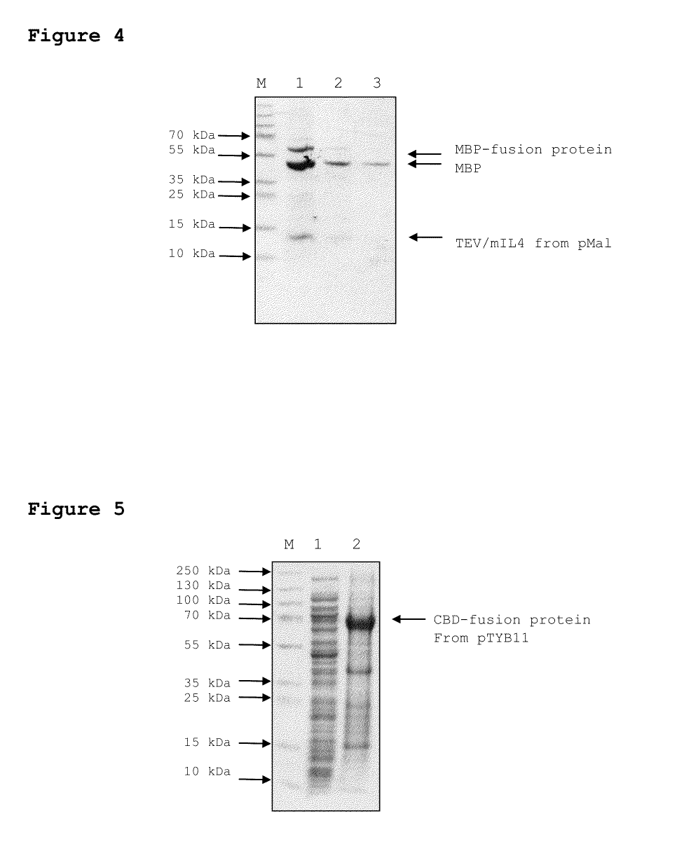

Expression of a maltose-binding-protein (MBP) fusion of mIL4 in pMalc2x is done according to the manufacturer's recommendations in E. coli XL10Gold results in good expression yield of soluble fusion protein. When cleaved with TEV protease the resulting free mIL4 protein cannot be detected in significant amounts (see also FIG. 4). The problem is most likely due to the disadvantageous ratio in molecular size of MBP and mIL4 and the obvious tendency of free mIL4QY to nonspecifically bind to surfaces, maybe due to insufficient folding and exposed hydrophobic patches.

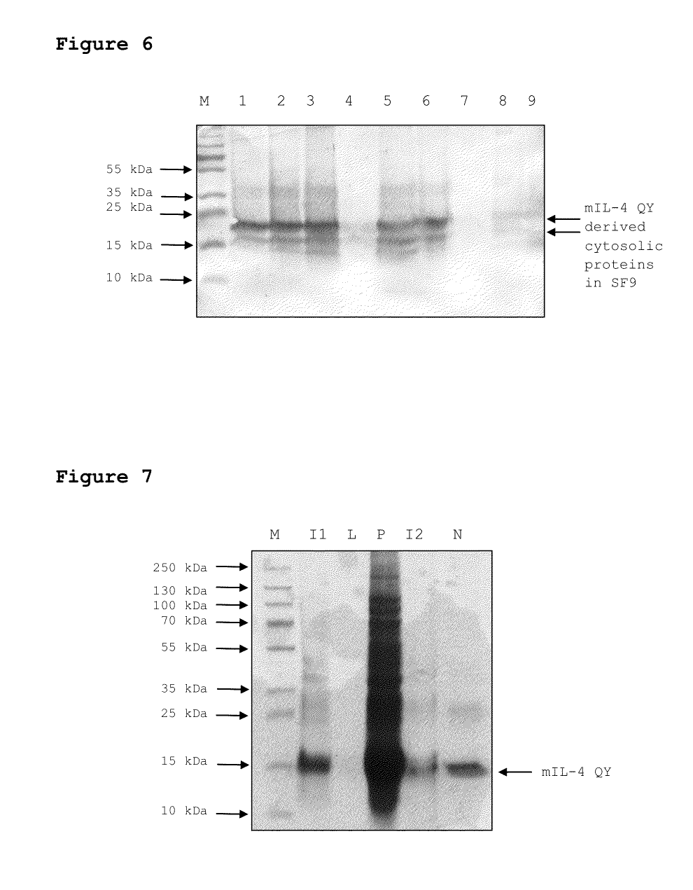

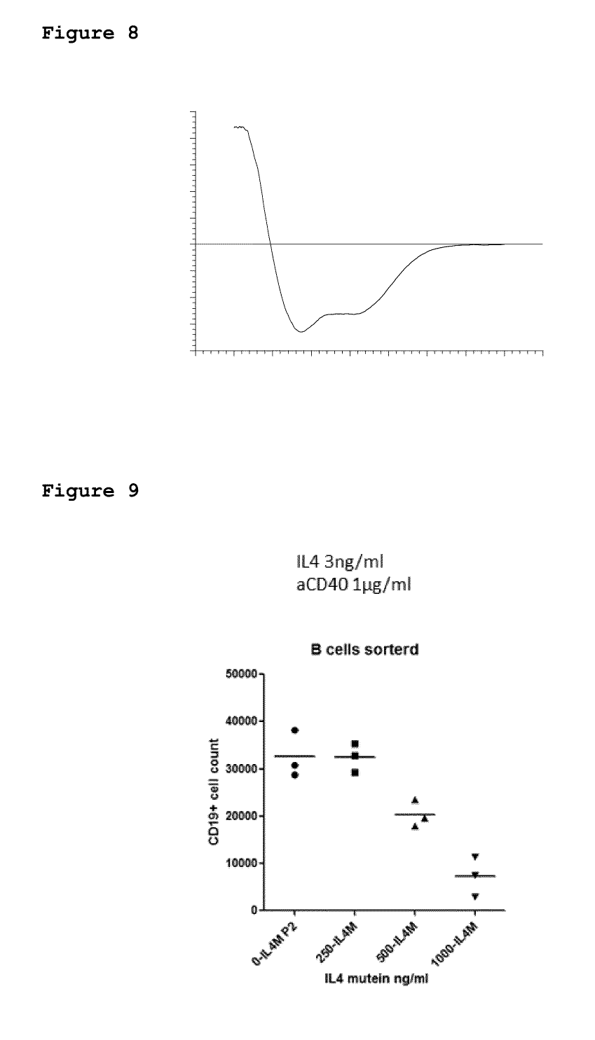

Expression of a chitin-binding-domain (CBD) fusion of mIL4 in pTYB11 is done according to the manufacturer's recommendations in E. coli BL21(DE3). When the cells are sonificated in chitin column buffer the fusion protein is found in the insoluble protein fraction (see also FIG. 5), making it impossible to purify without refolding. Expression of the pACgp67B construct is done in SF9 cells using the BaculoGold (Becton Dickinson) virus generation and amplification procedure. Test expressions are performed after 3 rounds of virus stock amplification and with different MOI, using dilutions of the virus stock. The result is the detection of only minor amounts of secreted mIL4 in the supernatant, utilizing an monoclonal anti-V5 antibody (Invitrogen). The majority of the protein is being detected in the cell lysate, showing only partial cleavage of the signal peptide (see FIG. 6).

Refolding from Inclusion Bodies.

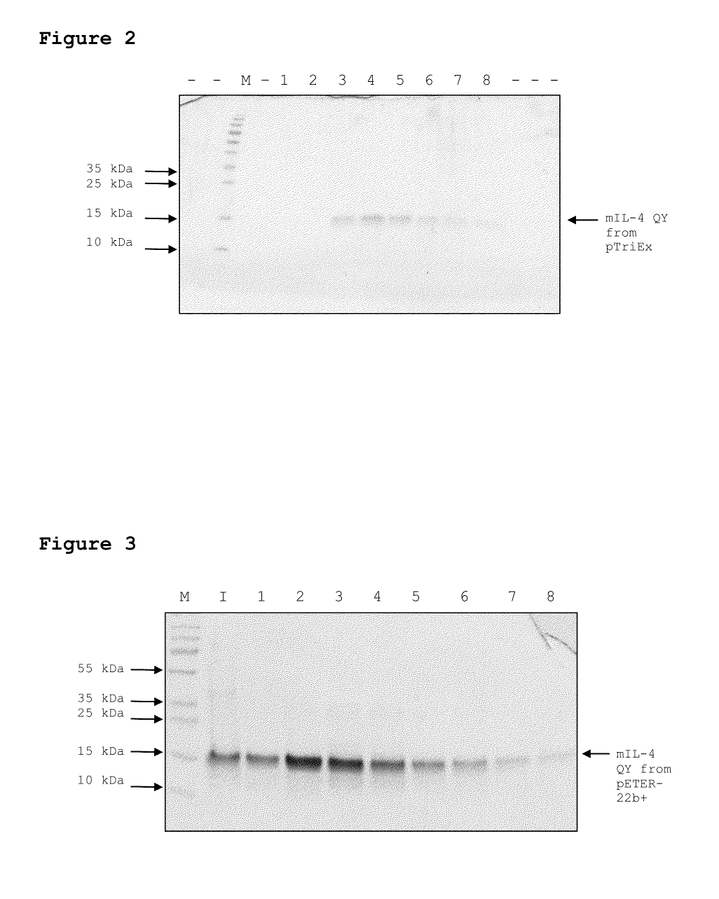

The cell pellet from pTriEx expression is either directly cooled on ice or thawed on ice and sonicated for 10 minutes at 30% duty cycle with power output setting 4 and a ¾″ sonotrode (Branson Sonifier 250). The solution is centrifuged at 11.000×g and 4° C. for 20 minutes. The supernatant is discarded and the pellet is resuspended in ⅓ volume lysis buffer with 2 M urea to wash the inclusion bodies. The suspension is centrifuged at 11.000×g and 4° C. for 20 minutes. The supernatant is discarded again and the pellet is again resuspended in lysis buffer with 2 M urea and centrifuged at 11.000×g and 4° C. for 20 minutes. The supernatant is discarded and the pellet is dissolved in ⅙ volume extraction buffer (6M guanidinium hydrochloride, 50 mM potassium phosphate, 1 mM reduced glutathione, pH 8.0). Shake for 10 minutes at room temperature. The sample is centrifuged at 11.000×g and 4° C. for 10 minutes. The pellet is discarded and the supernatant is loaded to a Ni-column (Pharmacia HiTrap™FF, 1 ml), equilibrated with extraction buffer, at approximately 1 ml per min. The column is washed with 5 volumes (5 ml) Wash buffer (7 M urea, 50 mM potassium phosphate, 100 mM sodium chloride, 1 mM red. glutathione, pH 6.8). The column is washed with 5× volume Unfolding buffer (7 M urea, 50 mM potassium phosphate, 100 mM sodium chloride, 1 mM red. glutathione, 0.1 mM oxidized glutathione, pH 6.8) and a linear gradient is applied, consisting of 50× volume to Refolding buffer (50 mM potassium phosphate, 100 mM sodium chloride, 1 mM red. glutathione, 0.1 mM oxidized glutathione, pH 6.8). The column is washed with 5× volume Refolding buffer and eluted with 5× volume Elution buffer (10 mM Tris, 250 mM imidazole, pH 8.0). The fractions are analyzed on 15% acrylamide gels and positive fractions are dialyzed against PBS (containing 5 mM magnesium chloride and 5 mM calcium chloride. The yield of the pTriEx vector construct in expression and refolding is found to best in the Tuner strain but generally to be low (see FIG. 2, therefore other constructs are tested. In terms of ease of expression and yield the pETER-22b+ construct is preferred.

Cells from the pETER-22b+ based expression are thawed and resuspended in 50 ml Tris buffer (50 mM Tris, pH 7.5; Sigma-Aldrich) and sonified on ice for 10 minutes (40% duty cycle, power output setting 4, and a ¾″ sonotrode; Branson Sonifier 250). The solution is centrifuged at 10.000×g and 4° C. for 30 minutes (Sorvall RC-5B). The pellet is resuspended in 45 ml Tris buffer with 1.5% Triton X-100 (Sigma-Aldrich). The resuspension is again sonified with above settings and incubated in a 50 ml tube on a roller overnight at 4° C. The solution is centrifuged again at 10.000×g and 4° C. for 30 minutes, the supernatant discarded and the pellet resuspended in Tris buffer. The solution is centrifuged again at 10.000×g and 4° C. for 30 minutes, the supernatant discarded and the pellet resuspended in 5 ml 1×PBS (phosphate buffered saline) containing 8 M urea, pH adjusted to 8.0. The solution was centrifuged again and the supernatant was loaded onto a Ni-affinity matrix column (Pharmacia, HiTrap™ Ni 1 ml) connected to an Äkta chromatograpy instrument (Pharmacia, Äkta Prime). The column is washed with 55 ml PBS+8 M urea and then changed to 160 ml PBS pH 8.0 without urea. Then the column is washed with 195 ml PBS pH 8.0 containing 10 mM imidazole and then eluted with 250 ml PBS containing 300 mM imidazole. The eluted fractions analyzed on coomassie stained PAGE are shown in FIG. 3.

An alternative refolding strategy is performed according to the published procedure by Levine et al. (1995). The purified inclusion bodies are dissolved in Extraction buffer A (50 mM CHES, 4 M guanidine hydrochloride, 500 mM NaCl, 50 mM DTT, pH 9.5) and left for 6 hours at room temperature. The solution is dialyzed against 15 volumes of disulfide slow formation buffer B (50 mM CHES, 4 M guanidine hydrochloride, 500 mM NaCl, 20 mM cysteine, pH 9.5) for 12 hours at room temperature. Then the dialysis buffer is exchanged to disulfide quick formation buffer C (50 mM CHES, 4 M guanidine hydrochloride, 500 mM NaCl, 20 mM cysteine, pH 9.5). After 12 hours it is changed to buffer D (50 mM CHES, 4 M guanidine hydrochloride, 500 mM NaCl, pH 9.5). The buffer is changed after 12 hours to fresh buffer D. The oxidized mIL4 is then dialyzed against refolding buffer E (50 mM CHES, 500 mM NaCl, pH 9.5) for 12 hours. Buffer E is renewed and dialysis continuous for another 12 hours. The salt is removed by dialysis against buffer F (50 mM CHES, pH 9.5). Especially during the last step a yellowish-white precipitate forms. After dialysis the solution is centrifuged at 10.000×g for 30 minutes at 4° C. to remove the insoluble precipitate. The refolded mIL4 is dialyzed against storage buffer G (50 mM acetic acid, pH 5). During dialysis significant amounts of a whitish precipitate form. This is removed again by centrifugation. An analysis of the precipitate reveals that it consists mainly of insoluble mIL4 protein. The total time frame of the refolding procedure according to Levine et al is around 4 days. In FIG. 7 the comparison of both refolding procedures is shown. It can be seen that most of the protein precipitates in the last step of the Levine et al. procedure and only a minor amount of protein remains soluble. The concentration of soluble mIL4 is higher in the Ni-matrix refolding strategy.

### Removal of the His-Tag.

The His-tag is preferably cleaved off overnight at 4° C. by incubation of fusion protein with recombinant TEV protease (His-tagged, Mobitec), according to the manufacturers' recommendation. Residual TEV protease can be removed by chromatography on Ni-affinity matrix. The His-tag is removed by passing the cleaved protein through Ni-matrix beads. The purified samples are dialyzed in a desired buffer and kept at 4° C. for short term storage or stored at −80° C. in the presence of 25% glycerol.

### Analysis of Correct Refolding.

The structural integrity of the murine IL-4 antagonist QY is analyzed by circular dichroism. The spectrum shows the same alpha-helical pattern as the comparable published spectrum of human IL4. It can be concluded that the analyzed refolded mIL4QY from the pETER-22b+ is in a uniform folded state.

Another functional test for correct folding is the cellular binding assay of murine IL-4 and the murine IL-4 antagonist QY to the murine IL-4 receptor. The functionality of murine IL-4 and the murine IL-4 antagonist QY is determined in a T cell proliferation assay (for details see Example 2).

### Example 2

### In Vitro Assays for Quantitative Determinations of Murine IL-4 and the Murine IL-4 Antagonist QY

In vitro assays for murine IL-4 and the murine IL-4 antagonist QY include an ELISA-type assay and cellular assays. The ELISA-type assay provides information about the concentration and integrity of the murine Il-4 epitope recognized by the anti-murine IL-4 antibody. Information about the structural-functional integrity of the murine IL-4 antagonist QY is obtained from a cellular binding assay analyzing binding of murine IL-4 and the murine IL-4 antagonist QY to the murine IL-4 receptor. The functionality of murine IL-4 and the murine IL-4 antagonist QY is determined in a T cell proliferation assay.

### ELISA Analysis.

The concentration of murine IL-4 or murine IL-4 mutants is determined by ELISA (Mouse IL-4 Sandwich ELISA cat#CMC0043, Invitrogen) according to the manufacturer's instructions.

### Cellular Binding Assay for Murine IL-4 and Murine IL-4 Antagonist QY.

Binding of murine Il-4 or the murine IL-4 antagonist QY to IL-4 receptor molecules on the surface of murine spleen cells or CTLL-2 cells is detected by FACScan analysis using a biotinylated anti-IL-4 antibody and fluorescent-labeled streptavidin. Murine CTLL-2 cells are IL-2-dependent and typically express 2000-5000 Il-4 receptors per cell.

Murine CTLL-2 cells, obtained from ATCC (ATCC TIB 214), are cultured in RPMI 1640 containing 8% fetal calf serum, 100 units/ml penicillin and 100 μg/ml streptomycin, supplemented with 100 ng/ml IL-2. For the cellular binding assay 5×10⁶ cells are incubated for 30 min at 37° C. in 150 μl of PBS (pH 7.4) containing 1% (w/v) BSA, 1×10⁻⁹ M recombinant murine (rmu) IL-4 or the murine IL-4 antagonist QY. The mixture is chilled to 4° C., washed once in a large volume of PBS (pH 7.4) containing 1% (w/v) BSA, resuspended in 150 μl of PBS (pH 7.4) and subjected to FACScan analysis.

### T Cell Proliferation Assay—CTLL-2 Cells.

Il-4 induced proliferation of CTLL-2 cells is determined by incorporation of [³H]thymidine as described (Duschl et al., 1992). The inhibitory effect of the murine IL-4 antagonist QY is determined by adding increasing amounts of the antagonist to IL-4.

CTLL-2 cells are incubated in 0.2 ml aliquots at a density of 5×10⁵/ml with log 2 dilutions of murine Il-4 for 3 days, before the amount of [³H]thymidine incorporated during the final 4 hr is determined. The concentration of IL-4 yielding half-maximal response is used for determining the inhibitory activity of the murine IL-4 antagonist QY. In these experiments, log 2 dilutions of the murine IL-4 antagonist QY are added to wild-type murine IL-4.

### T Cell Proliferation Assay—Murine Spleen B-Cells.

The proliferation assay is performed according to Hasbold et al (Eur. J. Immunol. 28:1040-1051, 1998). Naïve spleen cells from C57/BL/6 mice are cultured in cultivation medium (RPMI 1640+L-Glutamine (Gibco) with 1% glutamine, 1% penicillin-strepomycin, 10% fetal calf serum, 50 μM beta-mercatoethanol, 1% sodium pyruvate and 1% Non-essential amino acids (NEAA)) in CO₂-controlled sterile incubators in tissue culture plates (Nunc). 2*10⁶ total murine spleen cells are cultured per well in 200 μl medium. Cells are induced to proliferate with anti-CD40 (1 μg/ml) and murine recombinant IL-4 (3 ng/ml). IL-4QY mutein is added to the culture medium to inhibit the proliferation reaction. The enterotoxin concentration of the mutein is determined to be 0.01-0.02 ng/μg. The concentration of murine IL-4 and mIL-4 QY is chosen according to the kinetic data published in Grunewald et al (1997). 0, 250, 500 and 1000 ng/ml of mutein are added to the proliferation reactions. The proliferation is allowed to continue for four days. Cells are counted by FACS. B cells are specifically counted by labeling with anti-CD19 antibody.

An increasing inhibition of proliferation is detected between 250 and 1000 ng/ml of recombinant mIL-4 QY mutein, expressed in E. coli (pETER-22b+ construct) and refolded on Ni-matrix (see FIG. 9). The addition of different concentrations of mIL-4 QY mutein without IL-4 stimulation resulted in no significant change in B-cell numbers (data not shown).

### Example 3

### Synthesis and Characterization of Thermogelling PLGA-PEG-PLGA Hydrogels

The biodegradable triblock polymer described in this example consists of 30% PEG (1500), and a lactide/glycolide molar ratio of 15/1 (18.6/1 weight ratio). Synthesis of the triblock copolymer is performed according to published protocols (Quiao et al., 2005; WO 02/102309).

### Copolymer Synthesis.

Polyethylene glycol (PEG 1500) is purchased from Fluka, poly(DL-lactide) from Sigma, glycolide (1,4-Dioxane-2,5-dione) from Sigma, and stannous 2-ethylhexanoate from Aldrich.

A total of 25 g of DL-lactide, glycolide and PEG are used for polymerization (16.61 g DL-lactide, 0.89 g glycolide, 7.5 g PEG 1500). Under nitrogen atmosphere, PEG 1500 is dried in a round flask (equipped with a argon/vacuum inlet, under vacuum and stirring at 120° C. for 2 h. The lactide and glycolide is added under argon and 40 μl of Stannous 2-ethylhexanoate are added by syringe under stirring. Then the tube is sealed under argon. The sealed tube is immersed and kept in an oil bath thermostated at 150° C. After 8 h the tube was cooled to room temperature, and the product is dissolved in cold water. After completely dissolved, the copolymer solution is heated to 80° C. to precipitate the copolymer and to remove the water-soluble low-molecular weight copolymers and unreacted monomers. The supernatant is decanted, the precipitated copolymer is again dissolved in cold water followed by heating to induce precipitation. This process of dissolution followed by precipitation is repeated three times. Finally, the copolymer is dried under vacuum at room temperature until constant weight.

### Molecular Weight Determination.

The molecular weight of the copolymer is determined by gel permeation chromatography using polystyrene standards as described by Quiao et al. (2005).

### Measurement of Gelation Temperature.

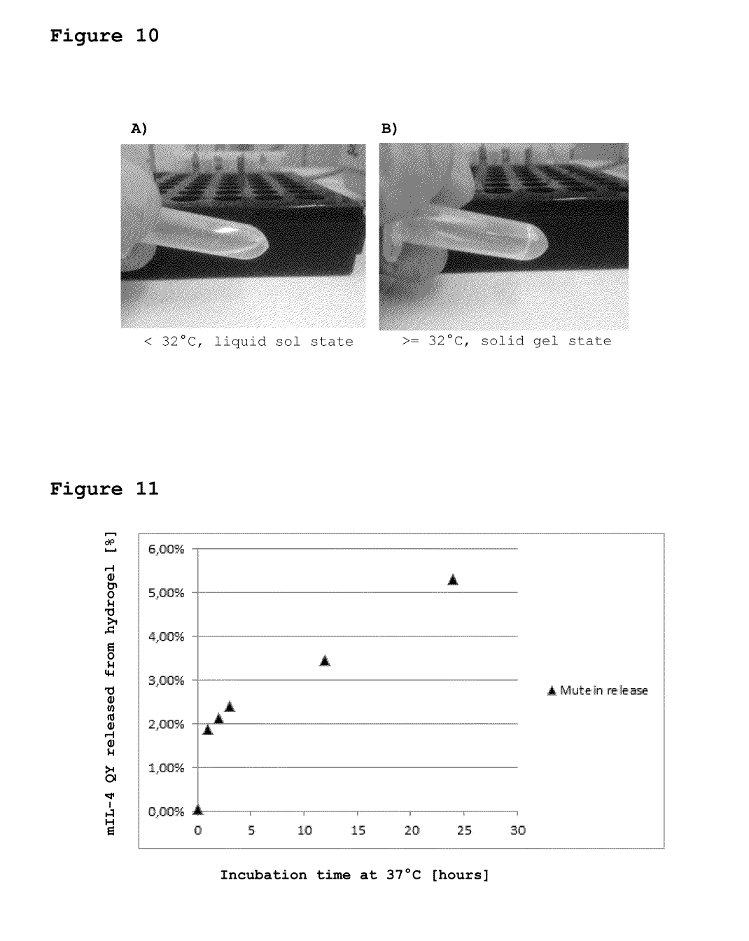

The gelation temperature is determined as described by Quiao et al. (2005). 200 μl of a 25% w/w solution of the hydrogel polymer in water is transferred to a 2 ml round-bottom reaction tube placed in temperature controlled incubator. The solution is heated at a controlled rate of 1° C. per measurement. At each temperature the tube is removed from the incubator and tilted towards 90 degree. When the solution appears gelled and solid the temperature read from the thermometer is determined as the gelation temperature (see also FIG. 10). The gelation temperature of the above described hydrogel is determined to be 32° C.

### Example 4

### In Vitro Degradation of Thermogelling PLGA-PEG-PLGA Hydrogels

The in vitro degradation behavior of the copolymer of Example 3 is evaluated by the mass loss and the molecular weight reduction with time upon incubation in phosphate buffered saline (PBS).

200 μl 25% Hydrogel in PBS are transferred into 2 ml tubes (empty weight determined) and gelled at 37° C. The gel is overlayed with 1.8 ml PBS. Several samples are incubated in PBS pH 7.4 at 37° C. under mild agitation in a water bath. At scheduled time intervals a sample is removed and the supernatant carefully aspirated. The tube with the solid gel residue is weighted (weight includes hydrogel, buffer salts and water) and then submitted to lyophilization. The tubes with dried gel are weighted (weight includes hydrogel and buffer salts) and the weight loss is calculated. It is assumed that 1 ml water contains 9.8 μg salts (1×PBS). Then the solid residues are dissolved in cold water and analyzed by gel permeation chromatography using polystyrene standards as described by Quiao et al. (2005). The gel described above swells during 6 hours by +23% and reaches a maximum at 48 hours of +41%, as determined by the differences in water content of gels. At the same time the gel degrades after 1 hour by 4%, 3 hours by 27%, 6 hours by 51% and after 48 hours reaches 55% weight loss, as determined by the differences in lyophilized hydrogel weight. The properties of the synthesised gel let assume that an elevated release of embedded small molecules over the first 6-12 hours by the combined action of swelling and degradation occurs, followed by an more constant release over the remaining time of slower degradation of the gel.

### Example 5

### In Vitro Release of the Murine IL-4 Antagonist QY from PLGA-PEG-PLGA/Murine IL-4 Antagonist QY Composites

The PLGA-PEG-PLGA triblock copolymer of Example 3 is dissolved at room temperature in distilled water to result in a 30% w/w stock solution. A 25% w/w hydrogel solution containing the murine IL-4 antagonist QY (1 μg/ml) is prepared by mixing 166.6 μl hydrogel stock 3.33 μl water, 20 μl 10×PBS and 10 μl mIl-4QY (200 μg/ml). Then 200 μl of the formulation is placed in a 2 ml reaction tube, incubated at 37° C. for 5 min until gelling, and overlayed with 1.8 ml pre-warmed 1×PBS pH 7.4. The vial is further incubated at 37° C. At specified sample collection times, the supernatant is removed and replaced by an identical volume of PBS pH 7.4 to maintain release conditions.

The amount of released murine IL-4 antagonist QY is determined by ELISA-type assay as described in Example 2. After 1 hour ˜2% of mutein is released. After 12 hours ˜3.5% and at 24 hours ˜5.5% of mutein is released by the above described hydrogel (see FIG. 11).

### Example 6

### Biodegradation and Release Characteristics of PLGA-PEG-PLGA/Murine IL-4 Antagonist QY Composites

To test the in vivo gel formation behavior and the release characteristics of composites of PLGA-PEG-PLGA hydrogels containing the murine IL-4 antagonist QY, the composites are injected subcutaneously into mice that have been anesthetized with ethyl ether. The resulting gel implants are then allowed to develop in vivo over the experimental period. At each of the post-injection sampling points, the mice are sacrificed and the gel implants are removed from the subcutaneous injection site. After determination of the volume of the removed gel implant, the remaining in vitro releasable amount of the murine IL-4 antagonist QY is determined as described Example 5.

Wild-type BALB/c mice (1 group: 16 mice) are analyzed for the in vivo gel formation behavior and the release characteristics. 25% (w/w) polymer containing increasing doses of the murine IL-4 antagonist QY (0.5 mg/ml, 1.0 mg/ml or 2.0 mg/ml) is used. The analysis is performed according to following schedule: implantation of 200 μl polymer composite on day 0, analysis of size and residual release in two mice on days 1, 2, 3, 4, 5, 10, 20 and 30.

### Example 7

### Effect of Co-Injection of Polymer-Embedded IL-4 Antagonist QY and Alum-Adsorbed Ovalbumin on the Humoral Immune Response in Mice

This example shows the effect of subcutaneously injected polymer-embedded IL-4 antagonist QY followed by the injection of alum-adsorbed ovalbumin (OVA) at the site of the gelled polymer composite, on the humoral immune response and development of allergic symptoms in mice.

Female wild-type BALB/c mice between 8 and 12 weeks of age are purchased from Charles River (Sulzfeld, Germany). The animals are kept under specific pathogen-free conditions and maintained on OVA-free diets. Ovalbumin (OVA) Grade V is purchased from Sigma, Imject Alum from Pierce/KMF, rat monoclonal antibodies against murine IgE and IgG1, and goat antisera against murine IgG2a, IgG2b, and IgG3 from BD Pharmigen.

### Preparation of Alum-Adsorbed Ovalbumin (OVA).

OVA (100 μg) is dissolved in 0.3 ml of PBS, pH 7.4, and mixed with 0.70 ml Imject Alum.

### Treatment Procedure.

Mice are injected subcutaneously 200 μl of the PLGA-PEG-PLGA triblock copolymer of Example 3 dissolved in distilled water containing increasing doses of the murine IL-4 antagonist QY (0.5 mg/ml, 1.0 mg/ml or 2.0 mg/ml). The final concentration of the copolymer in these experiments is 25% w/w. A control group receives the copolymer without embedded IL-4 antagonist QY. Before immunization with OVA analyses of OVA-specific antibodies, cytokine levels in serum and FOXP3 and GATA3 mRNA expression are performed. Six hours later, 100 μl alum-adsorbed OVA (10 μg in 100 μl of PBS/Imject Alum) are injected subcutaneously at the site of the gelled polymer composite (50 μl at each side of the gelled polymer). At day 5 after immunization with OVA, one group of mice (group 6) are injected again 100 μl of the PLGA-PEG-PLGA triblock copolymer of Example 3 containing the murine IL-4 antagonist QY. At day 10 after immunization with OVA, serum levels of OVA-specific antibodies are analyzed. Furthermore, immediate hypersensitivity in skin responses upon challenge via intradermal injection of OVA (3 mice of each group) and the development of anaphylactic shock upon i.v. injection of OVA (3 mice of each group) is assessed. Inguinal lymphnodes are used for a detailed analysis of T cell phenotypes.

Six groups of wild-type BALB/c mice (each group: 6 mice) are analyzed: group 1 (control 1): non-loaded polymer, mock immunization; group 2 (control 2): non-loaded polymer, immunization with alum-OVA; group 3: one injection of loaded polymer (100 μg antagonist), immunization with alum-OVA); group 4: one injection of loaded polymer (200 μg antagonist), immunization with alum-OVA; group 5: one injection of loaded polymer (400 μg antagonist), immunization with alum-OVA; and group 6: two injections of loaded polymer (total: 600 μg antagonist), immunization with alum-OVA.

### Analysis of Serum Levels of OVA-Specific Antibodies.

Mice are bled at day 0 before immunization with OVA and 10 days later. Murine anti-OVA IgE and IgG subclasses are determined by ELISA. Plates are coated with 10 μg OVA in 100 μl 0.1 M NaHCO₃ for 6 h at 37° C., followed by blocking with 200 μl 3% BSA in PBS, pH 7.4, for 2 h at 37° C. After washing, 100 μl of 1:40 serum dilutions with PBS, pH 7.4, containing 1% BSA are incubated overnight at 4° C. The amount of bound antibody is analyzed using horseradish peroxidise-conjugated antibodies with specificity for murine heavy chain classes (IgE, IgG1, IgG2a, IgG2b, IgG3). Analysis is performed at 405 nm in a microplate autoreader.

### Analysis of Serum Levels of Cytokines.

The cytokine supernatant is profiled using a panel of 27 cytokines including the Th2 cytokines IL-4, IL-5 and IL-13.

Analysis of FOXP3 and GATA3 mRNA Expression.

The read-out for T cell differentiation is FOXP3 and GATA3 mRNA expression, which are inversely regulated. In addition the release is followed in real-time using STAT6 responsive Luciferase systems reporter systems.

### Analysis of Immediate Cutaneous Hypersensitivity.

Active cutaneous anaphylaxis is tested by skin test after i.v. injection of 200 μl of 0.5% Evans Blue dye in PBS, pH 7.4. Thereafter, the skin of the belly is shaved and four injection sites are marked with a felt tip pen on the skin. Two of the marked sites are injected intradermally with 50 μl PBS, pH 7.4, containing 50 μg OVA, and the other two sites with protein-free PBS, pH 7.4. After 15 min, the mice were killed by cervical dislocation and the skin is stripped off for inspection of the injection sites. The intensity of blue patch formation on the dorsal side of the skin, resulting from fluid extravasation into the injection site upon mast cell degranulation, is scored by two independent observers. Reactions are rated as positive when the diameter of the blue patch exceeds 5 mm, which is pre-marked on the mouse skin. The intensity of bluing is rated in the following manner: 0=no blue patch formation; 1=slight bluing; 2=marked bluing; 3=strong bluing.

### Analysis of Anaphylactic Shock Symptoms.

Mice are injected i.v. 500 μg OVA in 200 μl 0.5% Evans Blue solution. After 15 min, symptoms of an anaphylactic shock are assessed including bluing (no=0; slight=1; strong=2), pilo erection (no=0; slight=1; strong=2), spontaneous activity (running around=0; sitting passively=1; lying=2), and responsiveness to external stimuli (running away upon touching=0; slight reaction=1; no reaction=2). The animals are considered to be in a state of shock if at least three of the four indicated symptoms are observed by two independent observers who are unaware of the sensitization status of each animal.

### Analysis of T Cell Phenotype in Inguinal Lymphnodes.

Right and left inguinal lymph nodes are removed and T cell phenotype is tested by in vitro re-stimulation with allergen (ELISPOT/Luminex).

### Example 8

### Specific Immunotherapy of OVA-Sensitized Mice with Alum-Adsorbed OVA and PLGA-PEG-PLGA/Murine IL-4 Antagonist QY Composites

This example shows the efficacy of immunotherapy in OVA-sensitized mice using subcutaneously injected polymer-embedded IL-4 antagonist QY and co-injected alum-adsorbed OVA at the site of the gelled polymer composite.

Female wild-type BALB/c mice between 8 and 12 weeks of age are purchased from Charles River (Sulzfeld, Germany). The animals are kept under specific pathogen-free conditions and maintained on OVA-free diets. Ovalbumin (OVA) Grade V is purchased from Sigma, Imject Alum from Pierce/KMF, rat monoclonal antibodies against murine IgE and IgG1, and goat antisera against murine IgG2a, IgG2b, and IgG3 from BD Pharmigen.

### Preparation of Alum-Adsorbed Ovalbumin (OVA).

OVA (100 μg) is solved in 0.3 ml of PBS, pH 7.4, and mixed with 0.70 ml Imject Alum (Pierce/KMF).

### Allergic Sensitization Procedure.

Mice are immunized three times by i.p. injection with 10 μg OVA in 200 μl of PBS, pH 7.4, mixed with 70 μl Imject Alum as adjuvant. The second i.p. injection is performed 7 days after the first injection and the third injection 14 days after the first injection.

### Immunotherapy.

One week after the third immunization with OVA, mice are subjected to two different modalities of immunotherapy, one based on conventional treatment with increasing doses of alum-adsorbed OVA, the other based on treatment with increasing doses of alum-adsorbed OVA in the presence of polymer-embedded murine IL-4 antagonist QY. Subcutaneous administration of increasing doses of alum-adsorbed OVA is performed at 10-day intervals. Subcutaneous administration of polymer-embedded murine IL-4 antagonist QY is performed at day 0 and day 5 within the 10-day intervals.

For immunotherapy of mi Gce with alum-adsorbed OVA only, 100 μl of PBS/Imject Alum containing increasing doses of OVA (5, 15, 30, 60, 90 μg) are injected subcutaneously at 10-day intervals.

For immunotherapy of mice with alum-adsorbed OVA in the presence of polymer-embedded murine IL-4 antagonist QY, 200 μl of distilled water containing the PLGA-PEG-PLGA triblock copolymer of Example 3 (15% w/w or 25% w/w) and 400 μg of the murine IL-4 antagonist QY, are injected subcutaneously first. After gelling of the polymer composite, 100 μl of PBS/Imject Alum containing increasing doses of OVA (5, 15, 30, 60, 90 μg) are injected subcutaneously at the site of the gelled polymer composite (50 μl at each side of the gelled polymer). At day 5 within each 10-day interval, 100 μl of distilled water containing the PLGA-PEG-PLGA triblock copolymer of Example 3 (15% w/w or 25% w/w) and 200 μg of the murine IL-4 antagonist QY, are injected subcutaneously at the site of the first injection. The injection procedure is repeated three times at a different subcutaneous site until the highest dose of alum-adsorbed OVA is injected.

At day 10 after the last immunotherapeutic treatment, serum levels of OVA-specific antibodies are analyzed. Furthermore, immediate hypersensitivity in skin responses upon challenge via intradermal injection of OVA and the development of anaphylactic shock upon i.v. injection of OVA are assessed.

Three groups of wild-type BALB/c mice (each group: 10 mice) are analyzed: group 1: immunization (3× alum-OVA), therapy with non-loaded polymer and PBS; group 2: immunization (3× alum-OVA), therapy with loaded polymer and alum-OVA; and group 3: immunization (3× alum-OVA), therapy with alum-OVA.

### Analysis of Serum Levels of OVA-Specific Antibodies.

Mice are bled at day 0 before immunization with OVA and 10 days later. Murine anti-OVA IgE and IgG subclasses are determined by ELISA. Plates are coated with 10 μg OVA in 100 μl 0.1 M NaHCO₃ for 6 h at 37° C., followed by blocking with 200 μl 3% BSA in PBS, pH 7.4, for 2 h at 37° C. After washing, 100 μl of 1:40 serum dilutions with PBS, pH 7.4, containing 1% BSA are incubated overnight at 4° C. The amount of bound antibody is analyzed using horseradish peroxidise-conjugated antibodies with specificity for murine heavy chain classes (IgE, IgG1, IgG2a, IgG2b, IgG3). Analysis is performed at 405 nm in a microplate autoreader.

### Analysis of Serum Levels of Cytokines.

The cytokine supernatant is profiled using a panel of 27 cytokines including the Th2 cytokines IL-4, IL-5 and IL-13.

Analysis of FOXP3 and GATA3 mRNA Expression.

The read-out for T cell differentiation is FOXP3 and GATA3 mRNA expression, which are inversely regulated. In addition the release is followed in realtime using STAT6 responsive Luciferase systems reporter systems.

### Analysis of Immediate Cutaneous Hypersensitivity.

Active cutaneous anaphylaxis is tested by skin test after i.v. injection of 200 μl of 0.5% Evans Blue dye in PBS, pH 7.4. Thereafter, the skin of the belly is shaved and four injection sites are marked with a felt tip pen on the skin. Two of the marked sites are injected intradermally with 50 μl PBS, pH 7.4, containing 50 μg OVA, and the other two sites with protein-free PBS, pH 7.4. After 15 min, the mice were killed by cervical dislocation and the skin is stripped off for inspection of the injection sites. The intensity of blue patch formation on the dorsal side of the skin, resulting from fluid extravasation into the injection site upon mast cell degranulation, is scored by two independent observers. Reactions are rated as positive when the diameter of the blue patch exceeds 5 mm, which is pre-marked on the mouse skin. The intensity of bluing is rated in the following manner: 0=no blue patch formation; 1=slight bluing; 2=marked bluing; 3=strong bluing.

### Analysis of Anaphylactic Shock Symptoms.

Mice are injected i.v. 500 μg OVA in 200 μl 0.5% Evans Blue solution. After 15 min, symptoms of an anaphylactic shock are assessed including bluing (no=0; slight=1; strong=2), pilo erection (no=0; slight=1; strong=2), spontaneous activity (running around=0; sitting passively=1; lying=2), and responsiveness to external stimuli (running away upon touching=0; slight reaction=1; no reaction=2). The animals are considered to be in a state of shock if at least three of the four indicated symptoms are observed by two independent observers who are unaware of the sensitization status of each animal.

### Analysis of T Cell Phenotype in Inguinal Lymphnodes.

Right and left inguinal lymph nodes are removed and T cell phenotype is tested by in vitro re-stimulation with allergen (ELISPOT/Luminex).

### Example 9

### Cloning, Expression and Purification of the Human IL-4 Antagonist RY

### Cloning of the Human IL-4 Antagonist RY (R121D/Y124D).

The sequence of the native mature human IL-4 protein is modified to include a TEV cleavable N-terminal 6× His-tag and the RY (R121D/Y124D) mutations (see SEQ ID 03). The protein sequence is translated into DNA using codon optimization for expression in prokaryotic E. coli cells (Puigbo et al, 2007; Grote et al, 2005). The resulting sequence (SEQ ID 04) is synthesized and amplified with primers 1 (SEQ ID 5, IL4his-for) and 2 (SEQ ID 6, HUIL4mut-back) using PCR with Pfu polymerase according to the manufacture and annealing temperatures rising from initial 5 cycles at 40° C. to 55° C. and 30 cycles total. The resulting PCF fragment is gel-purified in 1% agarose (GeneJET Gel Extraction Kit, Fermentas) and transferred to a restriction enzyme double digest containing Nde I and Xho I (Fermentas). The restriction digest is again gel-purified and the fragment is ligated into Nde I and Xho I cut pET-22b (Novagen) expression vector. The resulting clone is used as template to generate a pETER-22b+ construct, as described with murine IL-4 mutein QY.

### Expression.

E. coli BL21(DE3) (Novagen) containing the human IL-4 mutein expression vector pETER-22b+mIL4 RY are grown in 500 ml 2xYT medium until OD600=0.8-1 at 37° C. with shaking (220 rpm). Expression is induced with addition of 1 mM IPTG and cells are incubated at 37° C. with further shaking for 3 hours. The cells are centrifuged for 30 minutes at 5.000×g and 4° C. (Eppendorf 5804R centrifuge). The supernatant is discarded and the pelleted cells are resuspended and combined in 50 ml medium. The cells are again centrifuged for 15 minutes at 5.000×g and 4° C. (Eppendorf 5804R centrifuge). The supernatant is discarded and the cell pellet is frozen at −20° C.

Refolding from Inclusion Bodies.

Cells from the pETER-22b+ based expression are thawed and resuspended in 50 ml Tris buffer (50 mM Tris, pH 7.5; Sigma-Aldrich) and sonified on ice for 10 minutes (40% duty cycle, power output setting 4, and a ¾″ sonotrode; Branson Sonifier 250). The solution is centrifuged at 10.000×g and 4° C. for 30 minutes (Sorvall RC-5B). The pellet is resuspended in 45 ml Tris buffer with 1.5% Triton X-100 (Sigma-Aldrich). The resuspension is again sonified with above settings and incubated in a 50 ml tube on a roller overnight at 4° C. The solution is centrifuged again at 10.000×g and 4° C. for 30 minutes, the supernatant discarded and the pellet resuspended in Tris buffer. The solution is centrifuged again at 10.000×g and 4° C. for 30 minutes, the supernatant discarded and the pellet resuspended in 5 ml 1×PBS (phosphate buffered saline) containing 8 M urea, pH adjusted to 8.0. The solution was centrifuged again and the supernatant was loaded onto a Ni-affinity matrix column (Pharmacia, HiTrap™ 1 ml) connected to an Äkta chromatograpy instrument (Pharmacia, Äkta Prime). The chromatography is performed at 4° C. The column is washed with 55 ml PBS+8 M urea and then changed to 160 ml PBS pH 8.0 without urea. Then the column is washed with 195 ml PBS pH 8.0 containing 10 mM imidazole and then eluted with 250 ml PBS containing 300 mM imidazole. The protein is concentrated to approx. 2 mg/ml.

### Removal of the His-Tag.

The His-tag is preferably cleaved off overnight at 4° C. by incubation of fusion protein with recombinant TEV protease (His-tagged, Mobitec), according to the manufacturers' recommendation. Residual TEV protease can be removed by chromatography on Ni-affinity matrix. The His-tag is removed by passing the cleaved protein through Ni-matrix beads. The purified samples are dialyzed in a desired buffer and kept at 4° C. for short term storage or stored at −80° C. in the presence of 25% glycerol.

### Analysis of Correct Refolding.

The structural integrity of the murine IL-4 antagonist RY is analyzed by circular dichroism. The spectrum shows the same alpha-helical pattern as the comparable published spectrum of human IL4 or murine IL-4 mutein QY. It can be concluded that the analyzed refolded mIL4RY from the pETER-22b+ is correctly folded.

Another functional test for correct folding is the cellular binding assay of murine IL-4 and the murine IL-4 antagonist QY to the murine IL-4 receptor. The functionality of murine IL-4 and the murine IL-4 antagonist QY is determined in a T cell proliferation assay (for details see Example 10).

### Example 10

### In Vitro Assays for Quantitative Determinations of Human IL-4 and the Human IL-4 Antagonist RY

In vitro assays for human IL-4 and the human IL-4 antagonist RY include an ELISA-type assay and cellular assays. The ELISA-type assay provides information about the integrity of the human IL-4 epitope recognized by the anti-human IL-4 antibody. Information about the structural integrity of the human IL-4 antagonist RY is obtained from a cellular binding assay analyzing binding of human IL-4 and the human IL-4 antagonist RY to the human IL-4 receptor. The functionality of human IL-4 and the human IL-4 antagonist RY is determined in a T cell proliferation assay.

### ELISA Analysis.

The concentration of human IL-4 or human IL-4 mutants is determined by ELISA (LEGEND MAX™ Human IL-4 ELISA Kit, BioLegend GmbH) according to the manufacturer's instructions.

### Cellular Binding Assay.

Binding of human Il-4 or the human IL-4 antagonist RY to IL-4 receptor molecules on the surface of human Raji B-lymphoma cells is detected by FACScan analysis using a biotinylated anti-IL-4 antibody and fluorescent-labeled streptavidin.

Human Raji B-lymphoma cells are obtained from ATCC (ATCC CLL-86) and cultured in RPMI 1640 containing 10% fetal calf serum. The cells (5×10⁶) are incubated for 30 min at 37° C. in 150 μl of PBS (pH 7.4) containing 1% (w/v) BSA, 1×10⁻⁹ M human IL-4 or the human IL-4 antagonist RY. The mixture is chilled to 4° C., washed once in a large volume of PBS (pH 7.4) containing 1% (w/v) BSA, resuspended in 150 μl of PBS (pH 7.4) and subjected to FACScan analysis.

### T Cell Proliferation Assay.

Peripheral blood mononuclear cells are obtained from healthy donors, purified by Ficoll-Hypaque centrifugation (Pharmacia) and stored in aliquots at −80° C. Thawed cells are cultured for 7 days with 9 μg/ml phytohemagglutinine (PHA; HA-15, Wellcome) and comprise at this stage a high percentage of activated T-cells (PHA-blasts). Cells (10⁵/200 μl) are incubated with log 2 dilutions of human IL-4 for four days, before the cell count is determined. The concentration of IL-4 yielding half-maximal response is used for determining the inhibitory activity of the human IL-4 antagonist RY. In these experiments, log 2 dilutions of the human IL-4 antagonist RY are added to wild-type human IL-4.

### Example 11

### In Vitro Release of the Human IL-4 Antagonist RY from PLGA-PEG-PLGA/Human IL-4 Antagonist RY Composites

The PLGA-PEG-PLGA triblock copolymer of Example 3 is dissolved at room temperature in distilled water containing different concentrations of the human IL-4 antagonist RY (0.5 mg/ml up to 2.0 mg/ml) to make a 25% w/w solution. Then 200 μl of the formulation is placed in a 2 ml reaction vial, incubated at 37° C. for 5 min until gelling, and 1.8 ml of PBS pH 7.4 is added. The vial is shaken at 100 rpm at 37° C. At specified sample collection times, a sample is withdrawn and replaced by an identical volume of PBS pH 7.4 to maintain release conditions.

The amount of released human IL-4 antagonist RY is determined by an ELISA-type assay as described in Example 10. The structural integrity of released human IL-4 antagonist RY is determined by a cellular binding assay as described in Example 10. The functionality of released human IL-4 antagonist RY is determined by inhibition of IL-4-induced T cell proliferation as described in Example 10.

1. A pharmaceutical composition made of one or more preparations and comprising a physiologically effective dose of at least one IL-4 and/or IL-13 inhibitor and at least one allergens, and a matrix, wherein at least the inhibitor is solved or embedded, or whereon at least the inhibitor is coated or adsorbed, wherein the matrix is selected as to enable prolonged release of the inhibitor.

2. The composition according to claim 1, wherein the inhibitor is selected from the group consisting of antagonistic IL-4 and/or IL-13 derivatives, soluble cytokine receptor constructs with specificity for IL-4 and/or IL-13 and/or precursors thereof, monoclonal antibodies specific for IL-4 and/or IL-13 and/or their precursors and/or their receptors, binding fragments of such antibodies, proteinaceous constructs with specificity for IL-4 and/or IL-13 and/or their precursors and/or their receptors, and aptamers with specificity for IL-4 and/or IL-13 and/or their precursors and/or their receptors.

3. The composition according to claim 1, wherein at least two different inhibitors are comprised, wherein at least one inhibitor inhibits IL-4 and at least one other inhibitor inhibits IL-13, or wherein at least one inhibitor is comprised, which inhibits both, IL-4 and IL-13.

4. The composition according to claim 1, wherein the inhibitor has a plasma half-life of less than 7 days, preferably less than 1 day, most preferably less than 6 or 5 hours.

5. The composition according to claim 1, wherein the inhibitor is a human IL-4 mutant with one to three mutations preferably in at least one of the positions R121, Y124 and S125.

6. The composition according to claim 1, wherein the matrix is a biodegradable or biostable organic polymer, preferably biodegradable, more preferably thermogelling, in particular selected from the group consisting of styrene-isobutylene based block copolymer, olefin polymer, polyethylene, polypropylene, polyethylene oxide (PEO), polypropylene oxide (PPO), polyvinyl chloride, polytetrafluoroethylene, fluorinated ethylene propylene copolymer, polyvinyl acetate, polystyrene, poly(ethylene teraphthalate), polyurethane, polyurea, silicone rubbers, polyamides, polycarbonates, polyaldehydes, natural rubbers, polyester copolymers, styrene-butadiene copolymers ethylene vinyl acetate, polyorthoesters, polyiminocarbonates, aliphatic polycarbonates, polycaprolactone (PCL), poly-D,L-lactic acid (PDLLA), poly-L-lactic acid (PLLA), lactides of said lactic acids, polyphosphazenes polyethylene oxide, polyethylene teraphtholate (PET), polybutylene teraphtholate (PBT), PEBAX, Nylon, polyorthoesters, polylactic acids, polyglycolic acids, albumin, monomethoxypoly(ethylene glycol) (MPEG) or copolymers or mixtures of any of the above including poly(lactic-co-glycolic acid) (PLGA), copolymers of L-lactide and D,L-lactide, diblock copolymers consisting of MPEG and PCL, MPEG and (PCL-ran-PLLA), MPEG and PLGA, triblock copolymers consisting of PLGA-PEG-PLGA, PEG-PLGA-PEG, PEG-PCL-PEG, diblock and triblockpolymers consisting of PEO and PLLA, Poloxamers.

7. The composition according to claim 6, wherein the polymer is thermogelling and wherein the gelling temperature above which gelling commences is between 20° C. and 40° C., preferably between 25° C. and 30° C.

8. The composition according to claim 6, wherein the 90 weight-% degradation of the polymer in body environment and/or 90 weight-% release of the inhibitor from the polymer is completed within 1 to 10 days, preferably within 1 to 2 days.

9. The composition according to claim 1, wherein additionally an adjuvant is comprised, wherein the adjuvant is preferably selected from the group consisting of liposomes, aluminum salts, Montanide emulsions, granulocyte-macrophage colony stimulating factor (GM-CSF), adjuvant based on inulin, virosomes and polyphosphazenes, most preferably is selected from aluminum phosphate or aluminum hydroxide gels.

10. The composition according to claim 1, wherein the matrix is an adjuvant selected from the group consisting of liposomes, aluminum salts, Montanide emulsions, granulocyte-macrophage colony stimulating factor (GM-CSF), adjuvants based on inulin, virosomes and polyphosphazenes, most preferably is selected from aluminum phosphate or aluminum hydroxide gels.

11. The composition according to claim 1, wherein:
all components are mixed as a single preparation, or
the matrix and the inhibitor are mixed as a first preparation and the allergen and adjuvant are mixed as a second preparation.

12. The composition according to claim 1, wherein the composition is galenically prepared for administration by subcutaneous injection.

13. A use of a composition according to claim 1, wherein the preparation of a medicine for the treatment of immunological disorders, in particular allergic reactions, preferably wherein the medicine is administered in a therapeutically effective dose to a person in need of treatment of a immunological disorder.

14. A method for manufacturing a pharmaceutical composition according to claim 1, wherein the components are mixed with each other in an physiologically effective amount to obtain a composition wherein all components are mixed as a single preparation, or the matrix and the inhibitor are mixed as a first preparation and the allergen and adjuvant are mixed as a second preparation, and, wherein optionally galenic compounds are additionally admixed to the preparation.

15. The method for treatment of an immunological disorder, in particular an allergic reaction, wherein a composition according to claim 1 is administered in an therapeutically effective dose to a person in need of the treatment.


SEQUENCE LISTING (13 sequences)

SEQ ID NO: 1 - DNA, 614 bp, Homo sapiens
gatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 2 - protein, 153 aa, Homo sapiens [sequence omitted]

SEQ ID NO: 3 - protein, 150 aa, Homo sapiens
MGSSHHHHHHSSGLVPRGSHMHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMDEKDSKCSS

SEQ ID NO: 4 - DNA, 450 bp, Homo sapiens
atgggttcttctcaccaccaccaccaccactcttctggtctggttccgcgtggttctcacatgcacaaatgcgacatcaccctgcaggaaatcatcaaaaccctgaactctctgaccgaacagaaaaccctgtgcaccgaactgaccgttaccgacatcttcgcggcgtctaaaaacaccaccgaaaaagaaaccttctgccgtgcggcgaccgttctgcgtcagttctactctcaccacgaaaaagacacccgttgcctgggtgcgaccgcgcagcagttccaccgtcacaaacagctgatccgtttcctgaaacgtctggaccgtaacctgtggggtctggcgggtctgaactcttgcccggttaaagaagcgaaccagtctaccctggaaaacttcctggaacgtctgaaaaccatcatggacgaaaaagactctaaatgctcttct

SEQ ID NO: 5 - DNA, 29 bp, Homo sapiens
aaaaaaccatatgggttcttctcaccacc

SEQ ID NO: 6 - DNA, 29 bp, Homo sapiens
aaaactcgagttaagaagagcatttagag

SEQ ID NO: 7 - DNA, 580 bp, Mus musculus
atttgttagcatctcttgataaacttaattgtctctcgtcactgacggcacagagctattgatgggtctcaacccccagctagttgtcatcctgctcttctttctcgaatgtaccaggagccatatccacggatgcgacaaaaatcacttgagagagatcatcggcattttgaacgaggtcacaggagaagggacgccatgcacggagatggatgtgccaaacgtcctcacagcaacgaagaacaccacagagagtgagctcgtctgtagggcttccaaggtgcttcgcatattttatttaaaacatgggaaaactccatgcttgaagaagaactctagtgttctcatggagctgcagagactctttcgggcttttcgatgcctggattcatcgataagctgcaccatgaatgagtccaagtccacatcactgaaagacttcctggaaagcctaaagagcatcatgcaaatggattactcgtagtactgagccaccatgctttaacttatgaatttttaatggttttatttttaatatttatatatttataattcataaaataaaatatttgtataatgt

SEQ ID NO: 8 - protein, 140 aa, Mus Musculus
MGLNPQLVVILLFFLECTRSHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMQMDYS

SEQ ID NO: 9 - protein, 141 aa, Mus musculus
MGSSHHHHHHSSGLVPRGSHMHIHGCDKNHLREIIGILNEVTGEGTPCTEMDVPNVLTATKNTTESELVCRASKVLRIFYLKHGKTPCLKKNSSVLMELQRLFRAFRCLDSSISCTMNESKSTSLKDFLESLKSIMDMDDS

SEQ ID NO: 10 - DNA, 423 bp, Mus musculus
atgggttcttctcaccaccaccaccaccactcttctggtctggttccgcgtggttctcacatgcacatccacggttgcgacaaaaaccacctgcgtgaaatcatcggtatcctgaacgaagttaccggtgaaggtaccccgtgcaccgaaatggacgttccgaacgttctgaccgcgaccaaaaacaccaccgaatctgaactggtttgccgtgcgtctaaagttctgcgtatcttctacctgaaacacggtaaaaccccgtgcctgaaaaaaaactcttctgttctgatggaactgcagcgtctgttccgtgcgttccgttgcctggactcttctatctcttgcaccatgaacgaatctaaatctacctctctgaaagacttcctggaatctctgaaatctatcatggacatggacgactct

SEQ ID NO: 11 - DNA, 29 bp, Mus musculus
aaaactcgagttaagagtcgtccatgtcc

SEQ ID NO: 12 - DNA, 41 bp, Mus musculus
gatcccatgggcacattcacgggtgtgacaaaaatcatctg

SEQ ID NO: 13 - DNA, 38 bp, Mus musculus
gatcgcggccgctcagctgtcatccatatccatgatcg